(19) United States
(12) Patent Application Publication
(10) Pub. No.: US 2022/0009854 A1
(43) Pub. Date: Jan. 13, 2022

(54) A SERIES OF CATALYSTS FOR THE HYPERPOLARISATION OF SUBSTRATES

(71) Applicant: UNIVERSITY OF YORK, York North Yorkshire (GB)

(72) Inventors: Soumya Singha Roy, York North Yorkshire (GB); Wissam Iali, York North Yorkshire (GB); Ben Tickner, York North Yorkshire (GB); Simon Benedict Duckett, York North Yorkshire (GB)

(73) Assignee: UNIVERSITY OF YORK, York North Yorkshire (GB)

(21) Appl. No.: 17/291,851

(22) PCT Filed: Nov. 6, 2019
(86) PCT No.: PCT/GB2019/053146

§ 371 Date: May 6, 2021

(30) Foreign Application Priority Data
Nov. 7, 2018 (GB) 1818171.9

(51) Int. Cl. C07B 61/00; A61K 49/06

(52) U.S. Cl. CPC C07B 61/00; G01R 33/282; A61K 49/06

(57) ABSTRACT

There is described a method for the preparation of a hyperpolarised agent, wherein said agent comprises at least one —N⁻, —O⁻ or —S⁻ moiety (optionally protonated) and a secondary binding site; said method comprising: (i) preparing a fluid containing a polarisation transfer precatalyst and parahydrogen; (ii) introducing a co-ligand to interact with the transfer precatalyst to form a polarisation transfer catalyst; (iii) applying a magnetic field or radio frequency excitation to (ii), such that hyperpolarisation is transferred from parahydrogen to a target molecule; (iv) introducing a target molecule containing at least at least one —N⁻, —O⁻ or —S⁻ moiety, in conjunction with a secondary binding to form a hyperpolarised agent; wherein the co-ligand is selected from the group consisting of one or more of a sulfoxide, a thioester, a phosphine, an amine, CO, an isonitrile and a nitrogen heterocycle.

### FIELD OF THE INVENTION

The present invention relates to a method for the production of a hyperpolarised agent via hyperpolarisation transfer via a series of novel hyperpolarisation transfer catalysts.

More particularly, the present invention provides a method for the production of a hyperpolarised agent and the associated signal enhancement of ¹H, ¹³C, ³¹P, ¹⁹F, ²⁹Si, and ¹⁵N responses in a variety of species such as amines, amides, alcohols, sugars, carboxylic acids, oxalic acids, carbonic acids, phosphates, borates and pyruvate. The hyperpolarisation transfer described herein is generally based on the SABRE effect.

The present invention also provides novel hyperpolarisation transfer catalyst complexes and novel imaging media associated with the use of such novel hyperpolarisation transfer catalyst complexes.

### BACKGROUND OF THE INVENTION

Magnetic resonance imaging (MRI) is a technique based upon the science of nuclear magnetic resonance (NMR). MRI has become particularly attractive to physicians as images of parts of a patient's body thereof can be obtained non-invasively and without exposing the patient and the medical personnel to potentially harmful radiation such as X-rays.

Furthermore, due to its high quality images and good spatial and temporal resolution, MRI is a favourable imaging technique for imaging patients' soft tissue and organs. One of the main advantages of SABRE is that it achieves this result without the incorporation of p-H₂ into the substrate. This technique is effectively a form of catalysis which utilizes a suitable catalyst^{[4]}, to reversibly bind both H₂ (p-H₂) and the substrate in order to assemble a reaction intermediate in which polarisation is able to transfer, at low magnetic fields, from p-H₂ into the substrate.^{[5]}

NMR and MRI involve the detection of what can be viewed to be transitions of nuclear spins between an excited state and a ground state in an applied magnetic field. Because the energy difference between these states is relatively small, the usual Boltzmann distribution of chemically identical nuclei is such that at room temperature the populations of nuclear spin states which are in dynamic equilibrium are almost identical. Since the strength of the detected signal in magnetic resonance experiments is proportional to the population difference, NMR and MRI signals are typically weak.

The strength of detectable NMR signals can however be enhanced by hyperpolarising the magnetic nuclei. Hyperpolarisation, in this context, refers to a process in which a significant excess of magnetic nuclei are induced into a spin state. This results in a large increase in available signal due to the much larger inequality of populations across the energy levels that will ultimately be probed. In order for a hyperpolarised state to be useful, it is important that the spin state is sufficiently long lived to provide useful information, i.e. that the relaxation time of the spin state is ‘long’. The rules governing the relaxation rates of nuclear spins are complex, but known. It suffices to say that certain nuclei and spins systems have relaxation times which may extend from seconds to hours, days, months or even years.

There are a number of ways to induce certain nuclei into a hyperpolarised state. The simplest way is to cool the material to very low temperatures in the presence of a magnetic field, which will favour population of the lower energy state in which the spins of the nuclei are aligned with the applied magnetic field. This method is suitable for the production of hyperpolarised monatomic gases such as xenon or helium-3. The polarisation levels of these nuclei have also been increased via the use of laser-based technologies.

Hyperpolarisation aims to turn typically weak NMR and MRI responses into strong signals so that normally impractical measurements can be made. The parahydrogen based signal amplification by reversible exchange process (SABRE) has been used previously to hyperpolarise a range of agents that contain multiple bonds to nitrogen.

Nuclear magnetic resonance (NMR) reflects one of the most powerful methods to study materials while magnetic resonance imaging (MRI) plays a vital role in clinical diagnosis. However, the low sensitivity of these two techniques acts to limit their applicability, while adding substantially to cost. Remarkably, the hyperpolarisation method, dynamic nuclear polarisation (DNP) has been shown to improve the detectability of agents such as pyruvate, so that the MRI based diagnosis of disease through in vivo assessment of metabolism is possible. In contrast, easier to prepare parahydrogen (p-H₂), which exists in a pure nuclear spin state, was shown to enhance the strength of an NMR signal in 1987(1) but progress towards its clinical use has been limited. This reflects the fact it was originally used to detect chemically modified hydrogenation products, which acted to limit the range of organic materials it could work with.(2, 3) Only recently, in 2009, was a p-H₂ technique reported where the original chemical identity of the sensitised molecule was retained.(4) This approach is called Signal Amplification By Reversible Exchange (SABRE) and while it has proven to be highly successful for the hyperpolarisation of agents that contain multiple bonds to nitrogen such as nicotinamide(5), isoniazid(6), pyrazole(7), acetonitrile(8), operating on nuclei such as ¹H, ¹³C, ³¹P, ¹⁹F and ¹⁵N (5, 9-13), but there are many classes of molecule it currently fails to sensitise. This challenge is addressed here through a novel range of hyperpolarisation transfer catalysts that work more efficiently than many others.

The process of SABRE works by harnessing the latent polarisation of p-H₂ in the form of metal bound hydride ligands, and their hyperpolarisation is transferred into the magnetically active nuclei of a weakly bound substrate(14-16) via the small J-couplings that connect them, as quantified by Tessari.(17) Ligand exchange then enables the build-up of a pool of hyperpolarised substrate molecules in solution as detailed in Scheme 1.(18) Remarkably, ¹H polarisations of 60% have been achieved by this route, with ¹⁵N values of 50% seen. While in-high-field radio frequency (rf) transfer has achieved this effect (19) and superior sequences have been developed(20) we employ spontaneous low-field transfer here to create our hyperpolarised agents in a few seconds. Furthermore, as originally predicted,(14) very recent studies have established that SABRE can be used to produce hyperpolarised singlets(22) with magnetic-state-lifetimes that allow signals to be detected 15 minutes after their formation.

Hence the SABRE platform reflects a highly desirable route to hyperpolarisation and the extension here to dramatically improve the range of materials it works with is highly desirable.(22-25 & 38) One of the most effective precatalysts for this process has proved to be Ir(COD)(IMes)Cl (1) [where IMes=1,3-bis(2,4,6-trimethylphenyl) imidazole-2-ylidene, COD=cyclooctadiene] and it typically forms [Ir(H)₂(IMes)(substrate)₃]Cl (2) in protic solvents such as methanol⁸, although neutral Ir(H)₂(Cl)(IMes)(substrate)₂ (3) achieves similar results.⁹

### SUMMARY OF THE INVENTION

The initial purpose of the present study was to demonstrate that the target analyte pyruvate can be successfully hyperpolarised in the presence of a suitable metal complex. We report on the use of Ir(COD)(IMes)Cl (1) and a series of related precatalysts to achieve this. The precatalyst is activated by the addition of H₂, and the selected target substrate (e.g. pyruvate) now binds, but the presence of a specified co-ligand controls the outcome of this process. This combination of co-ligand/selected substrate is necessary to satisfy the 18-electron rule which leads to complex viability.

For pyruvate, the basis of this process is built around metal based Ir(H)₂(IMes)(CH₃COCOO), where the ligand CH₃COCOO⁻ has the potential to bind through one or two sites. Consequently, either one or two of the subsequent co-ligands can bind to iridium. Hence the active form of the catalyst can be [Ir(H)₂(IMes)(η¹-CH₃COCOO)(L)₂] (A) or [Ir(H)₂(IMes)(η²-CH₃COCOO)(L)] (B) and upon their creation they lead to different and controllable hyperpolarisation effects. The NHC, IMes and the co-ligand L can be changed to optimise this selectivity and the level of hyperpolarisation.

In this case, the former complex offers access to strongly hyperpolarized CH₃CO¹³COO⁻, CH₃¹³CO¹³COO⁻ and ¹³CH₃¹³CO¹³COO⁻ motifs while the latter form works well with the isotopologues CH₃¹³COCOO⁻ and CH₃¹³CO¹³COO⁻, ¹³CH₃COCOO⁻, ¹³CH₃¹³COCOO⁻, ¹³CH₃CO¹³COO⁻ and ¹³CH₃¹³CO¹³COO⁻. Their ²H labelled counterparts may also be used in the same way.

It is the identity of co-ligand L that is critical to this process, as if its binding is too strongly favoured then [Ir(H)₂(IMes)(L)₃]Cl will form preferentially (e.g. with pyridine) with very limited if any sensitisation of the ¹H and ¹³C NMR profiles of pyruvate. However, in conjunction with this process we discovered more generally a way to improve the sensitisation of other agents we exemplify pyruvate alongside these results.

Hence L is selected such that it meets the goal of weak binding. A particular example of ligand L is one or more sulfoxides, although other co-ligands are contemplated herein. Examples of sulfoxide co-ligands include a wide range of sulfoxides, such as, alkylsulfoxides, including, but not limited to, dimethylsulfoxide, diethylsulfoxide, dibutylsulfoxide and methylethylsulfoxide; and arylsulfoxides, including, but not limited to, diphenylsulfoxide, dibenzysulfoxide, phenylmethylsulfoxide, phenylethylsulfoxide, phenylvinyl sulfoxide and dimesityl sulfoxide (1,3,5-trimethyl-2-(2,4,6-trimethylphenyl)sulfinylbenzene); depending on the identity of the target analyte (illustrated above for pyruvate), the polarisation transfer mechanism, and the desire to create singlet polarisation in a suitable spin pair or Zeeman polarisation, the need for biocompatibility, the identity of the NHC and the choice of solvent.

It is also possible to use, temperature, the polarisation transfer-field, ²H labelled versions of these co-ligands and the NHC's, alongside their concentrations and those of the precatalyst, and the para-H₂ pressure, to improve the efficiency of the hyperpolarisation process.

This success in catalyst design represents an important breakthrough in enabling SABRE for biochemical analysis as it not only dramatically widens the range of agents it works with but improves results more generally.²⁹⁻³² The success in sensitising both the ¹H, ¹⁵N, ¹⁹F and ¹³C NMR profiles of this and other materials marks therefore a significant breakthrough in hyperpolarisation which has implications for both NMR and MRI.

Thus, according to a first aspect of the invention there is provided a method for the preparation of a hyperpolarised agent, wherein said agent comprises at least one —N⁻, —O⁻ or —S⁻ moiety (each of which may optionally be protonated) and at least one secondary binding site; said method comprising the steps of
(i) preparing a fluid containing a polarisation transfer precatalyst and parahydrogen;
(ii) separately or simultaneously introducing a co-ligand (L) to interact with the transfer precatalyst to facilitate the formation of polarisation transfer catalyst;
(iii) applying a magnetic field or radio frequency excitation to (ii), such that hyperpolarisation is transferred from parahydrogen to the target molecule when it is bound to the transfer catalyst;
(iv) separately or simultaneously introducing a target molecule, wherein said target molecule contains at least at least one —N⁻, —O⁻ or —S⁻ moiety, in conjunction with a secondary binding to form a hyperpolarised agent;
characterised in that the co-ligand is selected from the group consisting of one or more of a sulfoxide, a thioester, a phosphine, an amine, CO, an isonitrile and a nitrogen heterocycle.

The secondary binding site may vary depending upon the nature of the ligand. Thus, the secondary binding site may comprise a carbonyl function, a N-lone pair, or a hydroxyl group —OH. Examples of agents which comprise these types of secondary binding sites include, but shall not be limited to, pyruvate, salicylic acid, lactic acid, glycine, nicotinamide, etc. Alternatively, the binding site may consist of a single moiety such as —N⁻ or —O⁻ moiety (which may also be neutral or protonated) that binds through a lone pair of electrons to the catalyst, e.g. the metal complex.

The co-ligand will generally be bound to the precatalyst or the transfer catalyst.

According to this aspect of the invention the co-ligand (L) in this process interacts with the metal complex of the precatalyst or the transfer catalyst to facilitate the formation of species, such as, in the case of pyruvate, [Ir(H)₂(IMes)(η¹-CH₃COCOO)(L)₂] (A) and [Ir(H)₂(IMes)(η²-CH₃COCOO)(L)] (B) or, in the case of nicotinamide, [Ir(H)₂(IMes)(nicotinamide)(L)₂]Cl, or another salt, such as Br, or I, which act as the hyperpolarisation transfer catalysts. These co-ligands may already be contained in a precatalyst, such as [Ir(IMes)(COD)(DMSO)]⁺ or added to species such as Ir(IMes)COD(Cl). For the avoidance of doubt, “IMes” simply refers to a further ligand, in this case an NHC. The co-ligand (L) is fine-tuned, in conjunction with the NHC based carbene ligand (IMes) to optimise the efficiency of the transfer catalyst for a particular molecule e.g. pyruvate.

The transfer catalyst will generally be a magnetisation transfer catalyst.

The target molecule is a polarisable molecule, such as, but without limitation thereto, pyruvate, salicylic acid, lactic acid, glycine, nicotinamide, etc.

The target substrate can bind reversibly to the transfer catalyst during this process. By way of example, the NMR active nuclei of CH₃COCOO⁻ are polarised through the SABRE effect during this process.

In this aspect of the invention the active transfer catalyst, e.g. a metal complex, adds H₂ reversibly.

The initial H₂ addition/elimination step, or ligand loss steps, may be achieved by UV irradiation or may be thermal or photochemical in nature.

The hyperpolarisation may be achieved by polarisation transfer after, spin refrigeration, DNP, para-hydrogen induced polarisation (PHIP), SABRE or from a suitable molecule in a singlet state. However, in one particular aspect of the invention the hyperpolarisation is introduced by SABRE and thus, the transfer catalyst is a magnetisation transfer catalyst, especially a SABRE magnetisation transfer catalyst.

There are a number of ways to induce certain nuclei into a hyperpolarised state. The simplest way is to cool the material to very low temperatures in the presence of a magnetic field, which will favour population of the lower energy state in which the spins of the nuclei are aligned with the applied magnetic field. This method is suitable for the production of hyperpolarised monatomic gases such as xenon or helium-3. The polarisation levels of these nuclei have also been increased via the use of laser-based technologies.

In SABRE, a catalyst reversibly binds p-H₂ and the polarisable molecule to transfer dormant spin order from p-H₂ into the substrate via a scalar-coupling framework to the target molecule.

If there are two NMR active spins accepting polarisation, as exemplified by ¹³C₂ pyruvate, this will result in a singlet state in the polarisable molecule which will desirably be characterised by a long lifetime in a low magnetic field if there are two scalar coupled spin ½ nuclei present. Preferably, the resulting singlet state lifetime will be 20 seconds or more, preferably more than 20 seconds or more than 25 seconds or more than 30 seconds. The resulting singlet state lifetime may last one or more minutes.

When a SABRE type process is utilised as the method of hyperpolarisation, a SABRE hyperpolarisation transfer catalyst (e.g. [IrCl(COD)IMes] or a ²H-labelled counterpart or a related catalyst may be used to optimise the process in a suitable solvent with the selected singlet state derived agent.

H₂ or parahydrogen (p-H₂) gas may be the selected singlet state derived agent and after being added to the resulting system whilst agitating the system will activate the catalyst through a reaction whose speed may be enhanced by stirring, warming or shaking. Alternatively, the application of ultrasound may be used as a means of agitation. Hyperpolarisation transfer, by replacing the H₂ gas with p-H₂ may be performed to create a hyperpolarised transference complex whilst agitating the system as described herein. The addition of H₂ or parahydrogen (p-H₂) gas to the solvent may take place prior to the solvent system being agitated or may take place concurrent with agitation. Catalyst activation under parahydrogen may take place prior to the final hyperpolarisation transfer step or be part of the hyperpolarisation transfer step.

The catalyst and the hyperpolarisable target molecule may each contain appropriate ²H or Cl or O labels to maximise the relaxation times of the nuclear spins that are to be hyperpolarised (e.g. ¹H, ¹³C, ³¹P, ¹⁵N, ²⁹Si or ¹⁹F). The target molecule may contain appropriate ¹³C or ¹⁵N labelling to maximise the proportion of the target molecule that can be created in a hyperpolarised NMR visible form in conjunction with appropriate ²H, O or Cl labelling to extend their magnetic state lifetimes. Furthermore, the hyperpolarisable molecule may contain spin pairs of appropriate ¹H, ¹³C, ³¹P, ¹⁵N, ²⁹Si or ¹⁹F labels to enable the formation of long-lived states (singlet states) between the corresponding spin pairs (e.g. ¹H, ¹³C, ³¹P, ¹⁵N, ²⁹Si or ¹⁹F) within a molecular scaffold that contains appropriate ²H or Cl labelling to extend their lifetime. Long lived states may be created from a variety of spin pairs, including pairs comprising ¹H, ¹³C, ¹⁵N, ³¹P, ²⁹Si and ¹⁹F nuclei. The small molecule transference substrate will generally contain its spin ½ nuclei (e.g. ¹H, ¹³C, ³¹P, ¹⁵N, ²⁹Si or ¹⁹F) at the natural abundance level. In the case where the hyperpolarisable molecule contains pairs, these may be homo-nuclear or hetero-nuclear in nature. Examples, of such pairs include, but shall not be limited to ¹H/¹H, ¹H/¹³C, ¹H/¹⁹F, ¹H/¹⁵N or ¹³C/¹³C or any other combination of spin one half nuclei.

The co-ligand (L) and the other ligands surrounding the catalyst may include ²H labels in order to make the hyperpolarisation transfer process more selective and or efficient.

Hyperpolarisation will be transferred from parahydrogen into the polarisable target molecule in an optimised magnetic field to create a strongly hyperpolarised response. This may be subsequently converted into a singlet state across the spin-pair if desired. This conversion may occur spontaneously and optimised by selection of an appropriate magnetic field(s) for transfer or may be promoted by radio frequency excitation. It will be understood that a mixture of transfer catalysts may be included in the method of the invention to improve selectivity and allow mixtures to be examined.

The magnetic field can be changed to focus or improve the efficiency of hyperpolarisation transfer. The type of magnetic states required in this process may be ultra-low magnetic fields, e.g. <<1G (<10⁻⁶T) which can spontaneously hyperpolarise the said singlet state. A change in magnetic field can be used to control which substrates in a mixture gain signal in order to introduce selectivity, while varying the field during transfer step to enhance the signal from all substrates. Hence it will be possible to use this magnetic field to optimally polarise the MR active nuclei in the target substrate rather than the ligand L.

It will be understood that a polarisable molecule containing at least one —OH may comprise, individually or in combination, an OH moiety, such as methanol, ethanol, butanol, glucose, alkaloids, prostaglandins, or their salts e.g. NaOCH₃; NaOH; or a P—OH group, such as PO(OH)₃, or their salts e.g. PO(OH)₂(ONa), such as those P—OH groups found in DNA or adenosine triphosphate; or acid functionalities, such as HCOOH, CH₃COOH, CH₃CH₂COOH, CH₃COCOOH, or their salts e.g. NaOOCCH₃; and the like. These systems will result in complexes of type A.

However, if the OH moiety is supported by a second binding site such as CO as in the case of HOCH₂COMe or HOCH₂CH₂COMe or CH₃COCOOH then binding to form species of type B is also possible.

It will also be understood that the second binding site may reflect an N, NH, NH₂, S, or PO or SiO or SO or SO₂ or C═C or C≡C functional group which is capable of donating two electrons to the metal centre. Examples include, but are not limited to, HOCH₂CH₂NH₂, HOCH₂CH₂NHPh, HOCH₂CH₂NHPh; or amide, such as HOCH₂CH₂CONH₂ or NH₂CH₂COOH; and the like.

In one aspect of the invention the polarisable molecule contains at least one —OH moiety as herein defined.

In another aspect of the invention the polarisable molecule contains at least one —NH moiety as herein defined.

However, these simply reflect examples of ligand sites capable of donating an electron pair to the metal centre for interaction purposes and are not meant to be exhaustive.

When either the polarisable molecule or the target molecule comprises a molecule containing at least one —OH or —NH moiety, the pKa of the molecule, e.g. the amine or amide, can be varied to control the efficiency of binding alongside L and the NHC.

It will be understood that a mixture of target molecules may be included in the method of the invention.

It will also be understood that the protons on these agents can be removed by the addition of a base such as NaOH or Cs₂CO₃ to form the corresponding anion in order to further optimise lone pair availability.

Illustrative examples of target molecules which may be hyperpolarised via this route include, but shall not be limited to:
(i) R—OH or RO⁻ (wherein there is a suitable counter ion such as, but not limited to, Na⁺ or K⁺); wherein R represents alkylC₁₋₂₀, aryl, sugars, glycerol, vinyls, diols, cholesterol, choline, and the like;
(ii) R′COOH or R′COO⁻ (wherein there is a suitable counter ion such as, but not limited to, Na⁺ or K⁺); R′ represents H, alkyl_{C1-20}, aryl, vinyls, or any combination thereof, exemplified by acetic acid, acetate, pyruvate, pyruvic acid, an amino acid, a protein, an enzyme;
(iii) HOP(O)(R)(R′) wherein R and R′, which may be the same or different, each represents H, alkyl_{C1-20}, aryl, etc., such as part of a DNA base pair, strand or RNA or adenosine triphosphate;
(iv) HOBRR′ wherein R and R′, which may be the same or different, each represents H, alkyl_{C1-20}, aryl, etc., such as part of a borate;
(v) an inorganic or main group hydroxide such as Al(OH)₃ or Ca(OH)₂ and the like; or
(vi) a metal hydroxide such as ⁶LiOH, Al(OH)₃, related complexes containing hydroxide or amine ligands, where it extends to ²⁹Si, ⁷⁷Se, ¹¹³Cd, ¹⁹⁹Hg, ¹¹⁷Sn, ¹⁹⁵Pt, ²⁰⁷Pb, ⁵⁷Fe, ⁸⁹Y, ¹⁰⁹Ag and ¹⁸³W.

The target molecule may contain at least one —NH and may optionally comprise an amine or amide moiety. Thus, a polarisable molecule containing at least one —NH may comprise, individually or in combination, a primary, secondary or tertiary amine, such as NH₃, NH₂Ph, NH₂CH₂Ph, NH₂CH₂HCH₂CH₂Ph and related amines; or an amide, such as NH₂COCH₃ or NH₂CONH₂; and the like. An amine or amide can be used to control the efficiency of hyperpolarisation transfer.

Illustrative examples of target molecules which may be hyperpolarised via this route include, but shall not be limited to:
(i) NR′R″R″′ wherein R′, R″ and R′″, which may be the same or different, each represents H, alkyl_{C1-20}, aryl, base pair, etc. and combined in structures like glutamine, glutamate and GABA;
(ii) NR′R″COR″′ wherein R′, R″ and R′″, which may be the same or different, each represents R′, R″ or R′″═H, CH₃, alkyl, aryl, vinyl, or any combination exemplified by acetamide, urea, glutamine, glutamate, and the like;
(iii) carbamates and carbazides;
(iv) platinum derived cancer drugs, which include, but shall not be limited to cisplatin, carboplatin, nedaplatin, oxaliplatin, triplatin, satraplatin; and the like; and
(v) cancer drugs containing an acetamide group, which include, but shall not be limited to, taxanes such as paclitaxel, docetaxel, cabazitaxel; and the like.

According to another aspect of the invention the target molecule may comprise:
(i) HSR wherein R represents H, alkyl_{C1-20}, aryl, vinyls, or any combination thereof; and
(ii) thioamides, thioacids, thioureas and xanthates.

The hyperpolarisation transfer catalyst will usually comprise a transition metal complex, for example comprising a metal atom selected from, but not limited to, Ru, Rh, Ir, W, Pd and Pt. In a particular aspect of the present invention a hyperpolarisation transfer catalyst may comprise an iridium based catalyst whose key identity is controlled by the co-ligand.

Examples of preferred (SABRE) hyperpolarisation transfer precatalysts are thus described in our co-pending application No. PCT/GB2009/002860. Such catalysts include, for example, [IrCl(COD)(IMes)] and analogues thereof, (in which COD is cycloocta-1,5-diene). Alternatively, the (SABRE) hyperpolarisation transfer catalyst may comprise a ²H-labelled counterpart of [IrCl(COD)(IMes)] or a catalyst optimised to work in the non-aqueous phase with the selected substrate. Alternatively, the (SABRE) hyperpolarisation transfer catalyst may comprise of either of the two previous modifies in conjunction with a form like [IrL(COD)(IMes)]Cl which already contains L or a catalyst optimised to work in the non-aqueous phase with the selected substrate.

Generally, an iridium magnetisation transfer catalyst will include iridium with at least one N-heterocyclic carbene (NHC) ligand or phosphine.

Examples of such N-heterocyclic carbenes include, but shall not be limited to:

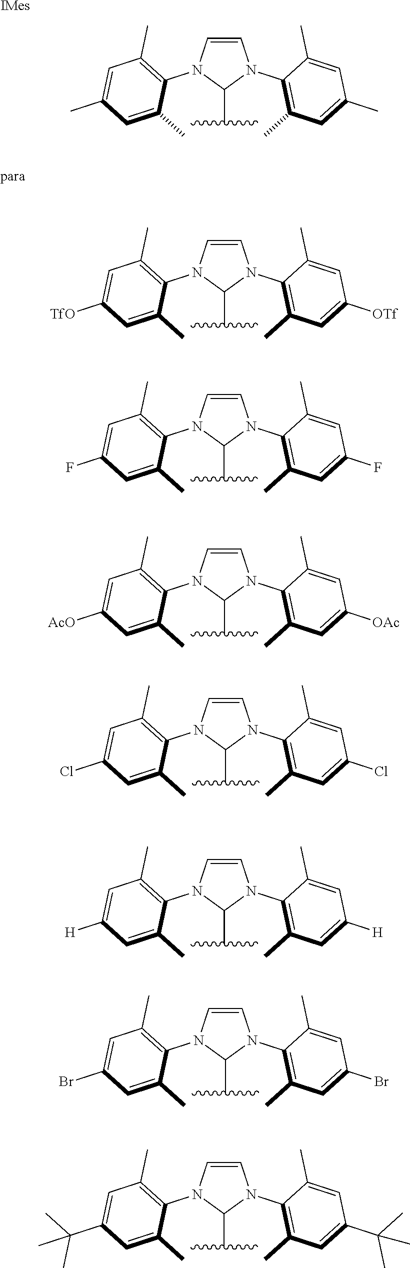

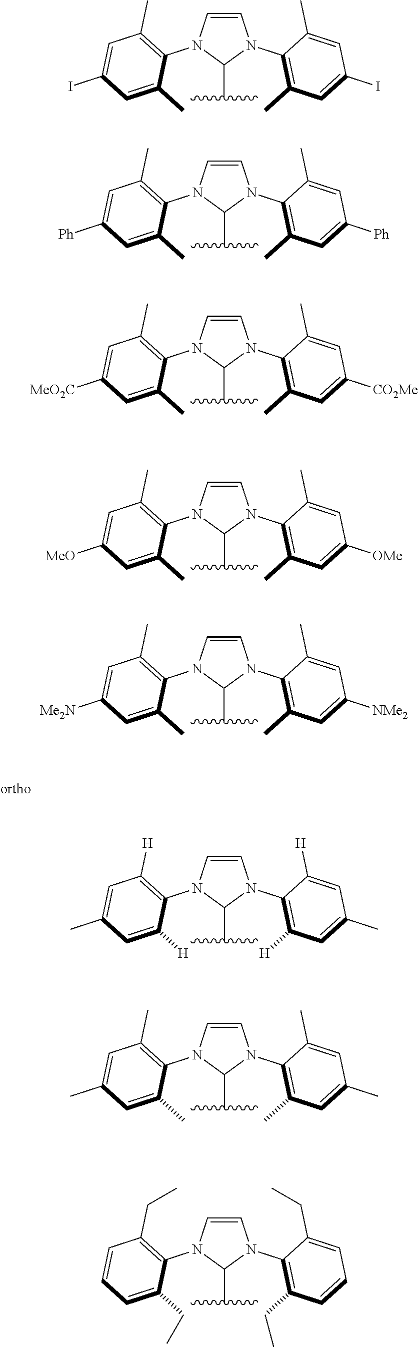

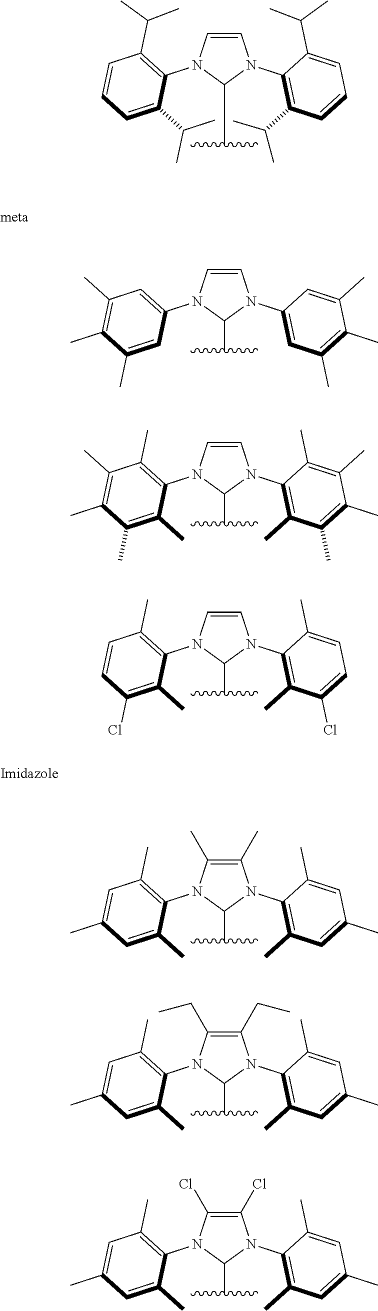

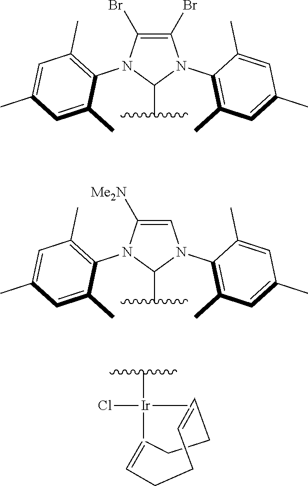

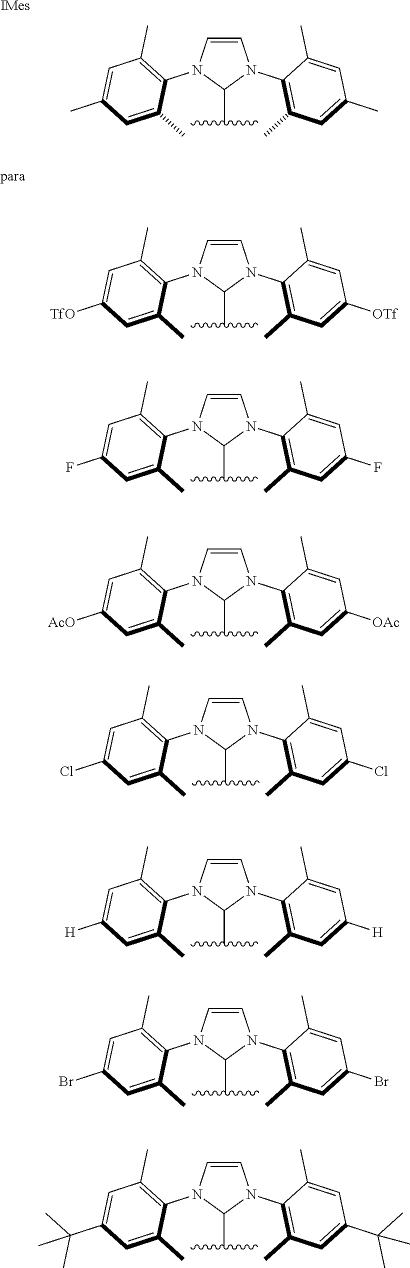

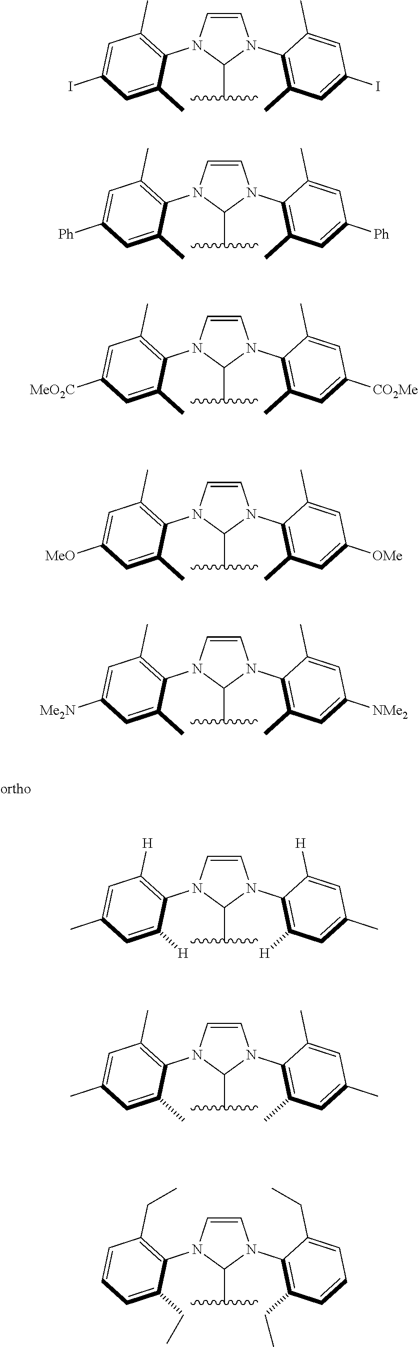

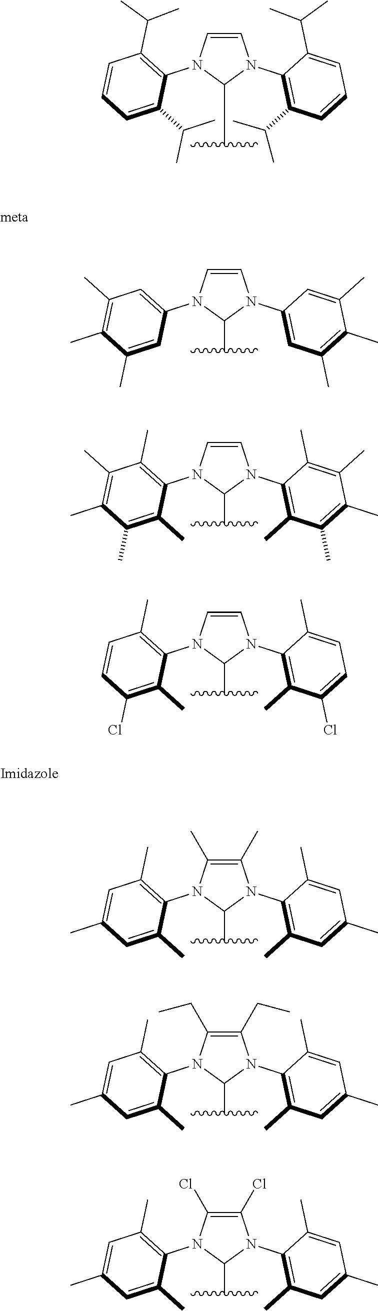

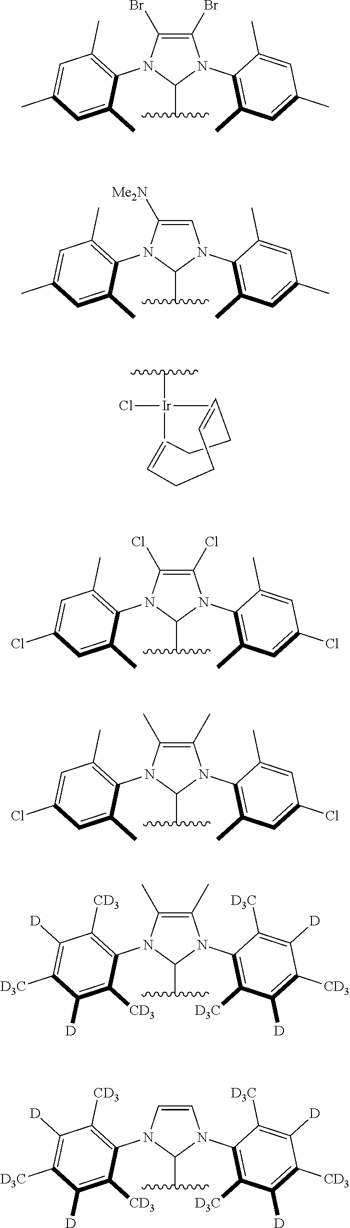

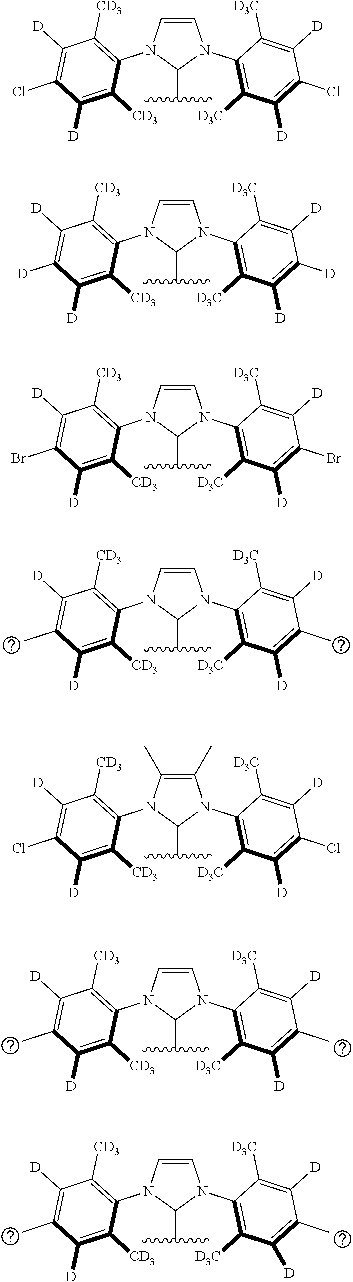

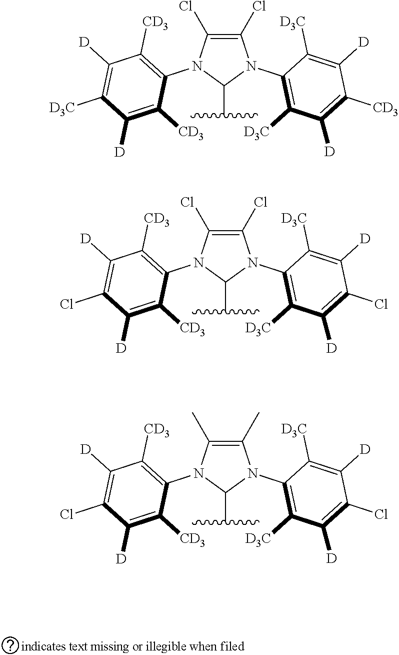

In the NHC version, the active form of these precatalysts will be based on [(Ir(H)₂(NHC)(L)]⁺ or [(Ir(H)₂(NHC)(L)₂]⁺ with the remaining metal coordination sites being occupied by the target molecule. The identity of the NCH and co-ligand L is varied to control the efficiency of hyperpolarisation transfer efficiency into the target molecule. For the phosphine versions, NHC is simply replaced by phosphine, e.g. PCy₃, PPh₃, PMePh₂ and the like.

The polarisation transfer catalyst may be designed to produce an optimal lifetime and coupling framework for hyperpolarisation transfer under these conditions. It will be understood that a mixture of transfer catalysts may be included in the method of the invention.

These species are often referred to as precatalysts because they are stable and become active during the catalytic process, in this case through their reaction with the small molecule substrate, the co-ligand L and H₂.

A variety of solvents may be used in preparing the fluid required for the method of the present invention. Such solvents will generally be organic solvents, e.g. a non-aqueous solvent; and may comprise polar, non-polar solvents, non-protic and protic solvents. Such solvents include, but shall not be limited to H₂O, CH₃OH, CH₃CH₂OH, CH₂OH, CH₂Cl₂, CHCl₃, THF, DMF, nitromethane, alkanes and aromatic hydrocarbons, such as benzene or toluene; the deuterated counterparts of any of the aforementioned solvents. Selection of an appropriate solvent may be used to control one or more of the steps herein defined in the method of the invention.

According to a further aspect of the invention a biphasic element may be introduced into the solvent in order to separate the hyperpolarised target molecule from the transfer catalyst.

The introduction of a biphasic element may comprise preparing a fluid containing two separate components, for example, wherein a first solvent is a polar solvent, e.g. water or saline and a second solvent is an immiscible co-solvent e.g. a non-polar solvent, such as, toluene, chloroform or dichloromethane. The ratio of solvent phases can be selected to:
(i) maximise the degree of target hyperpolarisation; and/or
(ii) maximise the speed of phase separation.

When required an aqueous solvent mixture combination may be used to maximise the relaxation time of the hyperpolarised target molecule in the solution by:
(i) employing D₂O;
(ii) employing a D₂O/H₂O mixture of suitable proportion e.g. 1:1; and/or
(iii) adding a further co-solvent to an appropriate aqueous phase such as ethanol or d₆-ethanol.

When SABRE hyperpolarisation is used, a SABRE hyperpolarisation transfer catalyst (e.g. [Ir(Cl(COD)(IMes)] or a ²H-labelled counterpart or one containing L or a catalyst optimised to work in the polar phase with the selected singlet state derived substrate).

When a mixed solvent system is used a solvent phase-separation promoter e.g. NaCl or NaO₂CCH₃ or NaOH or NaHCO₃ or Na₂CO₃ or ethanol, at a suitable concentration may be added to the system.

The concentration of the phase-separation promoter may be an amount suitable to:
(i) achieve physiological conditions;
(ii) vary the solutions pH to achieve optimal SABRE;
(iii) optimise organic phase extraction; and/or
(iv) optimise the speed of phase-separation.

Any known phase-separation promoter may be used. Desirably such a phase-separation promoter will be suitable for in vivo use and therefore should be suitable to achieve physiological conditions. In addition, the phase-separation promoter should be suitable to withstand variations in pH which may be desirable to achieve optimal SABRE. Selection of the phase-separation promoter may also be desirable to optimise organic phase extraction; and/or to optimise the speed of phase-separation.

Examples of phase-separation promoters include alkali metal salts, such as sodium or potassium salts; or alkaline earth metal salts, such as calcium. Alkali metal salts are preferred, such as NaCl, or NaO₂CCH₃, NaOH, NaHCO₃ or Na₂(CO₃). A further phase-separation promoter may comprise an alcohol such as ethanol.

The amount of phase-separation promoters may vary depending, inter alia, upon the nature of the phase-separation promoters, the nature of hyperpolarisation target, etc. When the aim is to create a biocompatible system, NaCl or KCl may be used as a phase-separation promoter to produce a saline or saline-like solution. Therefore, the amount of the phase-separation promoter may vary depending upon, inter alia, the nature of the phase-separation promoter. Generally, the phase-separation promoter may be from about 0.33% w/v to about 9% w/v. However, it will be understood by the person skilled in the art that more or less of the phase-separation promoter may be included, as required.

The hyperpolarisation transfer may be performed with p-H₂ to create a hyperpolarised target molecule whilst agitating the biphasic solvent as herein described.

An appropriate amount of time may be allowed to enable the two solution phases to separate.

The result of the hyperpolarisation process described herein is that the magnetic resonance signature of the target contains a hyperpolarised response in its ¹H, ¹⁹F, ¹³C, ³¹P, ²⁹Si and ¹⁵N nuclei. This is achieved through transfer of hyperpolarisation when the polarisable molecule is bound to the transfer catalyst, which locates a lone pair of electrons of the —OH, O⁻, N or —NH moiety within the bonding framework of complex A or B.

Furthermore, the use of ²H or ¹⁵N labelling in the polarisable molecule may be used to improve their relaxation times and increase the levels of detectable hyperpolarisation in them and the target molecule(s).

The polarisable molecule is then released from the metal in a hyperpolarised form and its hyperpolarised ¹H, ¹³C, ³¹P, ¹⁹F, ²⁹Si and ¹⁵N response can be detected.

In the presence of a target molecule hyperpolarisation can be transferred into the ¹H, ¹⁹F, ¹³C, ³¹P, ²⁹Si and/or ¹⁵N nuclei of the target molecule.

Through the process described herein the NMR or MR response of the target molecule can be increased so that it is readily detectable in a high resolution or imaging experiment.

Furthermore, the use of ²H or ¹⁵N labelling in the target molecule may be used to improve their relaxation times and increase the levels of detectable hyperpolarisation.

The target molecule will generally:
(i) contain spin ½ nuclei (e.g. ¹H, ¹³C, ³¹P, ¹⁵N, ²⁹Si or ¹⁹F) at the natural abundance level;
(ii) contain appropriate ²H, O or Cl labels to maximise the relaxation times of the nuclei spins that are to be hyperpolarised (e.g. ¹H, ¹³C, ³¹P, ²⁹Si N or ⁹F);
(iii) contain appropriate ¹³C or ¹⁵N labelling to maximise the proportion of the target that can be created in a hyperpolarised NMR visible form in conjunction with appropriate ²H or Cl labelling to extend their magnetic state lifetimes; and
(iv) contain pairs of appropriate ¹H, C, ³¹P, ¹⁵N, ²⁹Si or ¹⁹F labels to enable the formation of long-lived states (singlet states) between the corresponding spin pairs (e.g. ¹H, ¹³C, ³¹P, ¹⁵N or ¹⁹F) within a molecular scaffold that contains appropriate ²H, O or Cl labelling to extent their lifetime.

The hyperpolarisation target molecule may reflect a complex biomolecule containing exchangeable protons such as an enzyme, a protein, an alkaloid, an oligosaccharide or strand of DNA, RNA or adenosine triphosphate. The target biomolecule will become sensitised to NMR or MRI detection. This approach is therefore suited to the characterisation of large molecules and the probing of drug binding/active site conformations, dynamics and folding.

The use of L (e.g. DMSO, diethylsulfoxide, (etc.)) and their ²H or ¹³C labelled counterparts can be used to control the efficiency of hyperpolarisation transfer in the first step. This is a result of the metal complexes reactivity which can be optimised for specific solvent, cost, pressure of p-H₂ and time of activation.

The temperature can be changed to focus or improve the efficiency of hyperpolarisation transfer.

We note, more than one target may be present.

The transfer catalyst will usually comprise a transition metal complex, for example comprising a metal atom selected from, but not limited to, Ru, Rh, Ir, W, Pd and Pt. The transfer catalyst will usually comprise one or more ligands in addition to the ligand comprising the hyperpolarisable nuclei. These one or more other ligands may comprise organic or inorganic ligands and may be mono-, bi- or multidentate in nature. These one or more ligands may play a role in controlling the activity and stability of the metal centre. For example, the one or more ligands may comprise NHC ligands as herein described while the other ligand may be a sulfoxide.

In one embodiment, the transfer catalyst comprises one or more phosphine/co-ligand combinations in addition to the ligand to be hyperpolarised. The transfer catalyst may be attached to a solid support, for example a polymer support. Attachment will usually be made through a ligand which links the metal centre to the support. Suitable linkers are known in the art. For example, the linker may comprise one or more in-chain atoms selected from C, O, N, S, P and Si. The linker may comprise a siloxane moiety for attachment to the support and/or a phosphine moiety for attachment to the metal of the complex. In embodiments, the linker is a group of the following formula: —O—Si(OMe)₂-(CH₂)ₙ—P(Cy)₂-, wherein n is 0 upwards (e.g. 0, 1, 2, 3, 4, 5 or 6) and Cy is cyclohexyl.

In a further embodiment, the NHC or phosphine and co-ligand are linked together and form what is known as a chelate. This can be achieved via appropriate substitutions and the NHC/phosphine and co-ligand. Both cis and trans spanning may be induced by changing the length of the spacer. In this case the pre-catalyst is preassembled to include the co-ligand L.

For in vivo use an in-line UV probe may be used, if desired, to establish that the concentration of the catalyst is sufficiently for in vivo injection. This makes full use of the fact that the catalyst is no longer present and therefore unable to promote the relaxation of the agent, thereby maximising longevity of the resulting hyperpolarised signal.

For systems where the catalyst concentration remains too high, a catalyst deactivator may be added. Examples of suitable catalyst deactivators include, but shall not be limited to a chelating ligand, such as, bipyridyl, EDTA and dimethylglyoxime. A catalyst deactivator can be added to facilitate catalyst transfer.

An appropriate delivery device may be used to procure the hyperpolarised target molecule for detection by NMR or MRI which can facilitate some or all of the following: Using an appropriate delivery device to procure the hyperpolarised agent for detection by NMR or MRI which will facilitate some (all) of the following:
(i) after an appropriate amount removing a hyperpolarised sample from the aqueous phase;
(ii) using UV monitoring to assess suitability immediately prior to sample removal or after sample removal;
(iii) using pH monitoring to assess suitability immediately prior to sample removal or after sample removal;
(iv) employing filtration to achieve sterility after sample removal;
(v) injecting or transporting the sample into a target for subsequent detection by NMR or MRI, where the target might be a suitable sample tube, an animal or a human.

According to a further aspect of the invention there is provided a method of producing a hyperpolarised imaging medium, said method comprising the steps of
(i) preparing a fluid containing a polarisation transfer precatalyst and parahydrogen;
(ii) separately or simultaneously introducing a co-ligand (L) to interact with the transfer precatalyst to facilitate the formation of polarisation transfer catalyst, wherein the co-ligand is selected from the group consisting of one or more of a sulfoxide, a thioester, a phosphine, an amine, CO, an isonitrile and a nitrogen heterocycle;
(iii) applying a magnetic field or radio frequency excitation to (ii), such that hyperpolarisation is transferred from parahydrogen to the target molecule when it is bound to the transfer catalyst;
(iv) separately or simultaneously introducing a target molecule, wherein said target molecule contains at least at least one —N⁻, —O⁻ or —S⁻ moiety, in conjunction with a secondary binding to form a hyperpolarised agent;
(v) optionally separating the hyperpolarised target molecule(s) to provide a hyperpolarised target molecule imaging medium; and
(vi) completing NMR or MRI measurements on the system prior to repeating the process for signal averaging.

In a preferred aspect of the invention the imaging medium comprises a solution of a target molecule in a saline solution of a hyperpolarised target molecule.

According to this aspect of the invention the pharmaceutically acceptable formulation comprises a solution of a hyperpolarised target molecule, e.g. in a saline solution, for use as an imaging medium wherein said hyperpolarised target molecule is prepared by proton exchange from a hyperpolarised molecule containing at least one —OH, —NH or —SH moiety, said method comprising the steps of
(i) preparing a fluid containing a polarisation transfer precatalyst and parahydrogen;
(ii) separately or simultaneously introducing a co-ligand (L) to interact with the transfer precatalyst to facilitate the formation of polarisation transfer catalyst;
(iii) applying a magnetic field or radio frequency excitation to (ii), such that hyperpolarisation is transferred from parahydrogen to the target molecule when it is bound to the transfer catalyst;
(iv) separately or simultaneously introducing a target molecule, wherein said target molecule contains at least at least one —N⁻, —O⁻ or —S⁻ moiety, in conjunction with a secondary binding to form a hyperpolarised agent;
characterised in that the co-ligand is selected from the group consisting of one or more of a sulfoxide, a thioester, a phosphine, an amine, CO, an isonitrile and a nitrogen heterocycle.

The “proton exchange” may include establishment of a hydrogen bonding interaction between the polarisable molecule and the target molecule during the hyperpolarisation transfer step.

The hyperpolarisation may utilise parahydrogen enhanced hydride ligands of the transfer catalyst.

In this aspect of the invention in the pharmaceutically acceptable formulation the target molecule may include:
(i) R—OH or RO⁻ (wherein there is a suitable counter ion such as, but not limited to, Na⁺ or K⁺); wherein R represents alkyl_{C1-20}, aryl, sugars, glycerol, vinyls, diols, cholesterol, choline, and the like;
(ii) R′COOH or R′COO⁻ (wherein there is a suitable counter ion such as, but not limited to, Na⁺ or K⁺); R′ represents H, alkyl_{C1-20}, aryl, vinyls, or any combination thereof, exemplified by acetic acid, acetate, pyruvate, pyruvic acid, an amino acid, a protein, an enzyme;
(iii) HOP(O)(R)(R′) wherein R and R′, which may be the same or different, each represents H, alkyl_{C1-20}, aryl, etc., such as part of a DNA base pair, strand or RNA; or
(iv) HOBRR′ wherein R and R′, which may be the same or different, each represents H, alkyl_{C1-20}, aryl, etc., such as part of a borate; and
(v) an inorganic or main group hydroxide such as Al(OH)₃ or Ca(OH)₂ and the like.
(vi) A metal hydroxides such as ⁶LiOH, Al(OH)₃, related complexes containing hydroxide or amine ligands, where it extends to ²⁹Si, ⁷⁷Se, ¹¹³Cd, ¹⁹⁹Hg, ¹¹⁷Sn, ¹⁹⁵Pt, ²⁰⁷Pb, ⁵⁷Fe, ⁸⁹Y, ¹⁰⁹Ag and ¹⁸³W.

Preferably the target substrate is pyruvate.

We have demonstrated how it is possible to use parahydrogen to sensitise the MR response of a range of molecules that contain the four common and very important functional groups NH₂, OH, NH₂CO, COOH, COO⁻ and P(O)(OH)₃ in this way. We achieve this by taking a sulfoxide such as dimethylsulfoxide and reacting it with [IrCl(COD)(IMes)] (1) [where IMes=1,3-bis(2,4,6-trimethylphenyl) imidazole-2-ylidene, COD=cycloocta-1,5-diene] and p-H₂ alongside the target, such as pyruvate. Hence the active form of the catalyst will be [Ir(H)₂(IMes)(η¹-CH₃COCOO)(L)₂] (A) or [Ir(H)₂(IMes)(η²-CH₃COCOO)(L)] (B) and upon its creation lead to different and controllable hyperpolarisation effects in pyruvate of the other target.

For dimethylsulfoxide, [Ir(H)₂(IMes)(η²-CH₃COCOO)(DMSO)] rapidly forms in methanol or dichloromethane solution. At 243 K, in methanol-d₄, this complex exists in a number of isomers with one detailed below. In the schematic depiction (Scheme 2) of the SABRE hyperpolarization process wherein para-hydrogen (p-H₂) is used to hyperpolarize pyruvate by reference to the isotopologues 1-4 and an iridium catalyst (one of three geometric isomers shown).

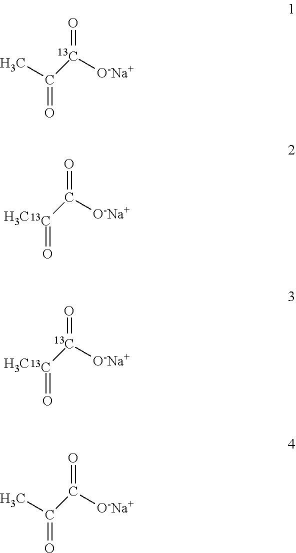

Because of the asymmetry of pyruvate, the resulting spin system in the catalyst must reflect a situation with two inequivalent hydrides regardless of their exact ligand arrangement.

At very low field it might therefore be expected that an [AA′B] spin system approximation results in the catalyst. Hence the propagation of hyperpolarisation from its hydride ligands into either ¹³C nuclei can be modelled. This process predicts the optimum polarisation transfer field is such that Bₜᵣₐₙₛ equals (−J_{HH}+J_{CC}+J_{HC})/Δγ. Here, J_{HH} corresponds to J-coupling between the hydrides ligands, J_{CC} is the ¹³C₂-coupling of the pyruvates, and J_{HC} (=(J_{HC}+J_{H′C}+J_{HC′}+J_{H′C′})/4) denotes the combination of all four hydride-carbon cross-couplings during the momentary substrate-catalyst association. Δγ (γ_{H}−γ_{C}) is the difference in magnetogyric ratios of proton and carbon. On this basis, transfer will be optimised into 1 at ±9 mG and 2 at ±5 mG. This is 100 times lower than the Earth's magnetic field and requires screening via a mu-metal shield in conjunction with a field top-up solenoid. In addition, a transfer null is predicted at 0 mG for both as a consequence of the mismatch between propagating terms.

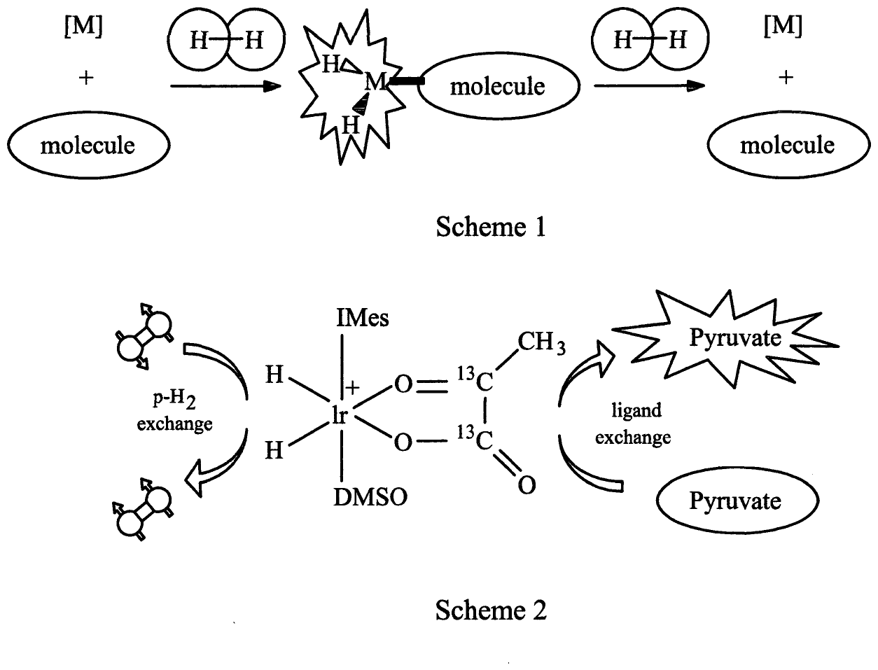

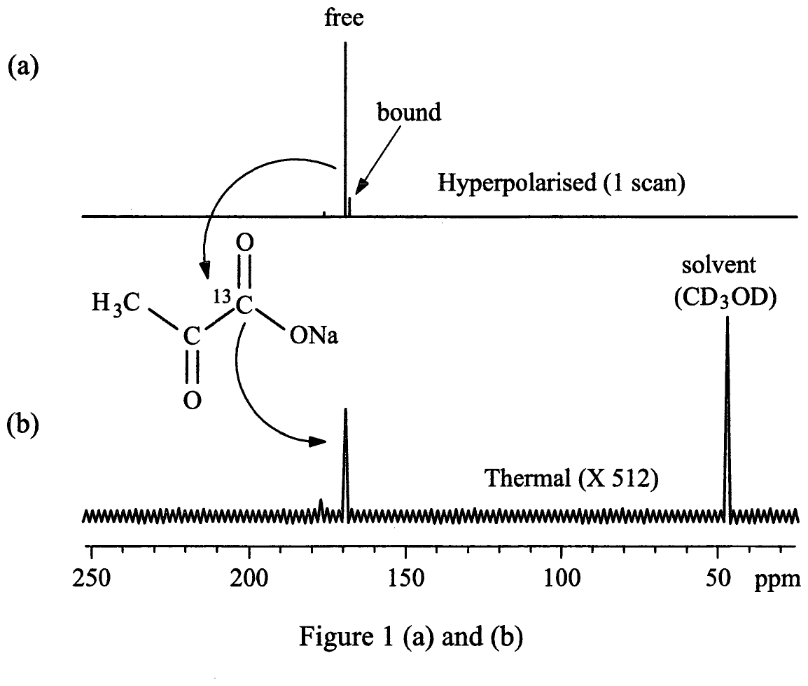

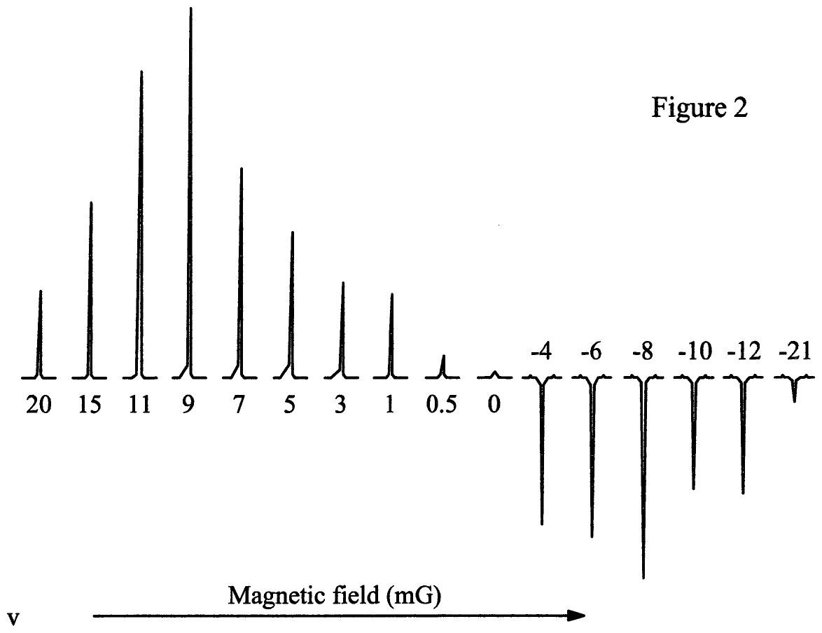

When a sample of sodium pyruvate-1-¹³C (1) is examined with p-H₂ the predicted propagation of hyperpolarisation from the hydride ligands into its ¹³C nuclei results. FIG. 1 shows the result of this process for 1 after transfer at 9 milli Gauss (mG) using precatalyst Ir(Cl)(COD)(IMes). The sample was located in the Earth's magnetic field which was screened via a mu-metal shield in conjunction with a field top-up solenoid. FIG. 2 shows the resulting efficiency versus field plot over the field range +20 to −21 mG to confirm the validity of our hyperpolarisation transfer condition which is indeed maximised at 9 mG. A periodic variation in amplitude and phase results such that both can be controlled by this setting. A ¹³C polarisation efficiency level of ca. 1% in this isotopologue was obtained in 20 seconds when the ratio of iridium precatalyst to 1 was 1:8 and the p-H₂ pressure 3 bar used.

The in-high-field relaxation time for the hyperpolarised C₁ resonance was then determined by a series of sequential low-tip measurements in a similar way to those previously obtained by DNP. A value of 32.5±4.7 s was obtained which compares to the corresponding non-hyperpolarised value of 35.4±0.5 s.

When the pyruvate sample was changed to 2 where the ¹³C label is now at C2 (2), the labelled resonance again becomes strongly hyperpolarised with the corresponding maximum polarisation value now being ca. 0.6%. This is achieved optimally after transfer at 5 mG rather than 9 mG for a similarly concentrated sample. The reduced efficiency can be attributed to the smaller J_{H13C} transfer coupling and shorter spin lifetime (see Table 1).

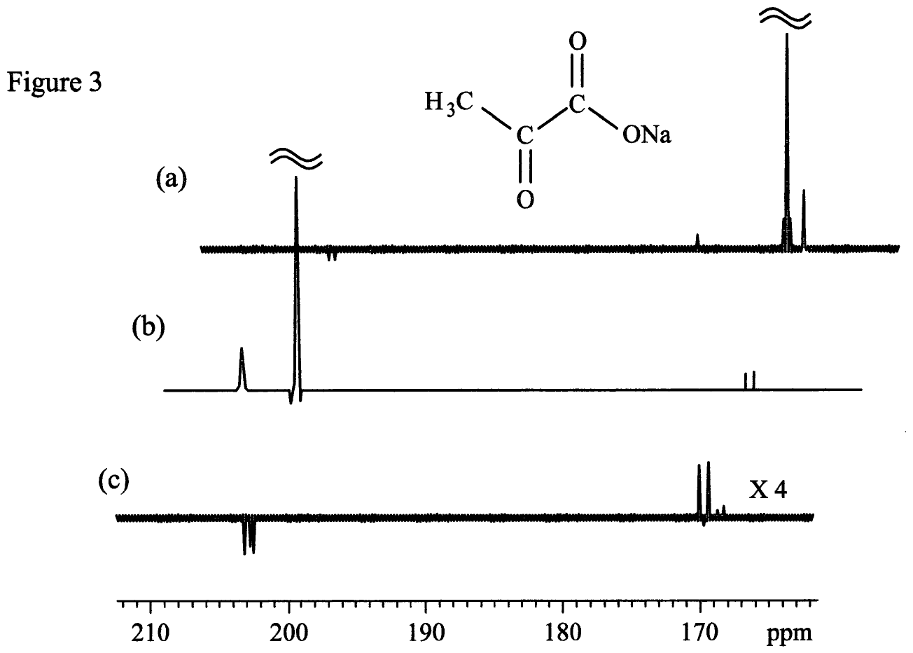

Closer examination the associated hyperpolarised ¹³C NMR spectra for samples 1 and 2 reveal further NMR peaks for bound pyruvate and the minor Na¹³CO₂¹³COMe isotopologue (3) at δ 170 ppm and δ 203 ppm respectively. These results are illustrated in FIG. 3. This form is present at just 1.1% of the level of 1 or 2 and confirms the impressive nature of the associated signal amplification. In addition, signals for the corresponding isotopologue with a ¹³C₃ (signal at δ 25.8 ppm) group could be seen; this signal was most readily detectable after transfer at 1-10 mG. The NMR behaviour of isotopomer 3 was rigorously probed and is described later.

The hyperpolarised responses of 1 and 2 were used to rapidly determine the associated ¹³C nuclei relaxation time by the standard low tip angle method. Values of 32.5±4.7 s and 18.2±3.0 s were obtained respectively The thermal T₁ values of the respective spins of 1 and 2 were measured by standard inversion recover experiments and calculated to be 37.0±0.5 s and 20.4±0.5 s respectively. This slight mismatch in the numbers can be attributed to the measurement procedures of small but constant low tip-angle detection method as pointed out earlier. All these measurements were carried out in the same sample volume in the presence of active catalysts. Comparing these numbers with reference to DNP-pyruvate results, we can conclude that the presence of the SABRE catalyst therefore does not significantly change these results which differ from the situation reported for many reported ¹H responses with SABRE. This suggests that the chemical exchange rate is slow in conjunction with the small J_{HC} values. The associated nuclear spin state lifetimes, under several conditions, and their hyperpolarisation levels are summarized in Table 1.

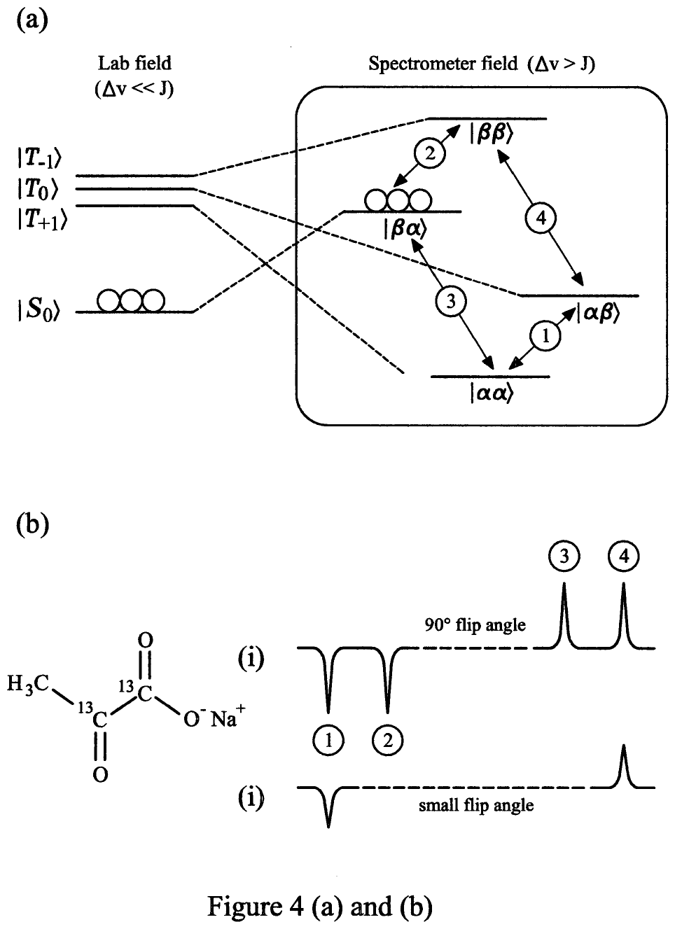

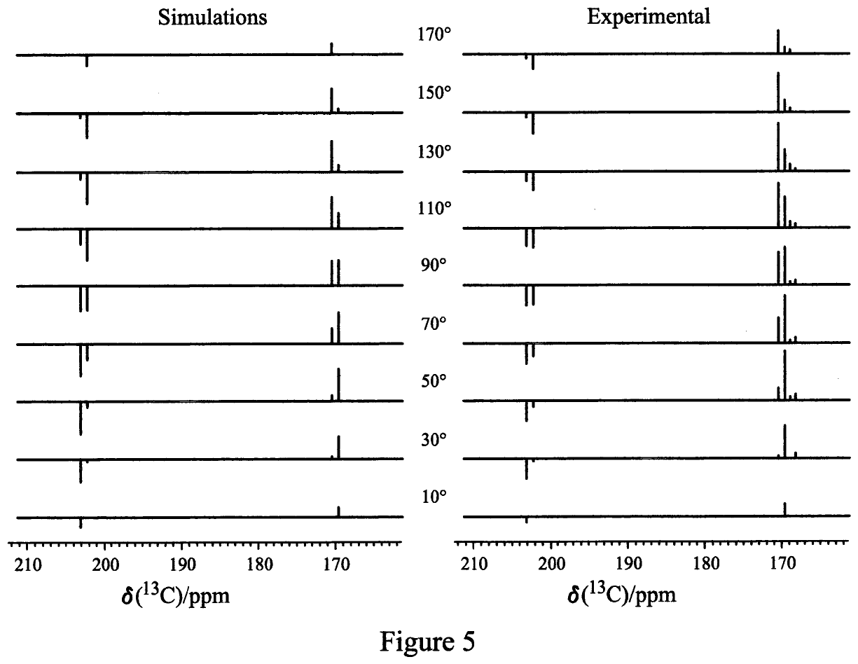

In contrast to the situation with 1 and 2, isotopomer 3 still gives a detectable response after transfer at 0 G. This is because the metaldihydride-Na¹³CO₂COMe complex that results now reflects a low field [AB]₂ spin system. Theoretical analysis under these conditions suggests that a ¹³C₂ singlet state readily forms across the range 0 G-1 kG. The results of this prediction could be easily confirmed experimentally by monitoring the effects of excitation angle on the resulting signal profile which produces a unique non-linear response (FIG. 4 for rational). A sample of 3 was therefore prepared and hyperpolarised as a function of polarisation transfer field. The resulting experimental NMR spectra are detailed in FIG. 5 alongside their simulated counterpart. A close fit between experiment data and simulation results. The singlet state is formed with amplitude 1.75% of the Zeeman polarisation that was observed for 3 at 11.75 T.

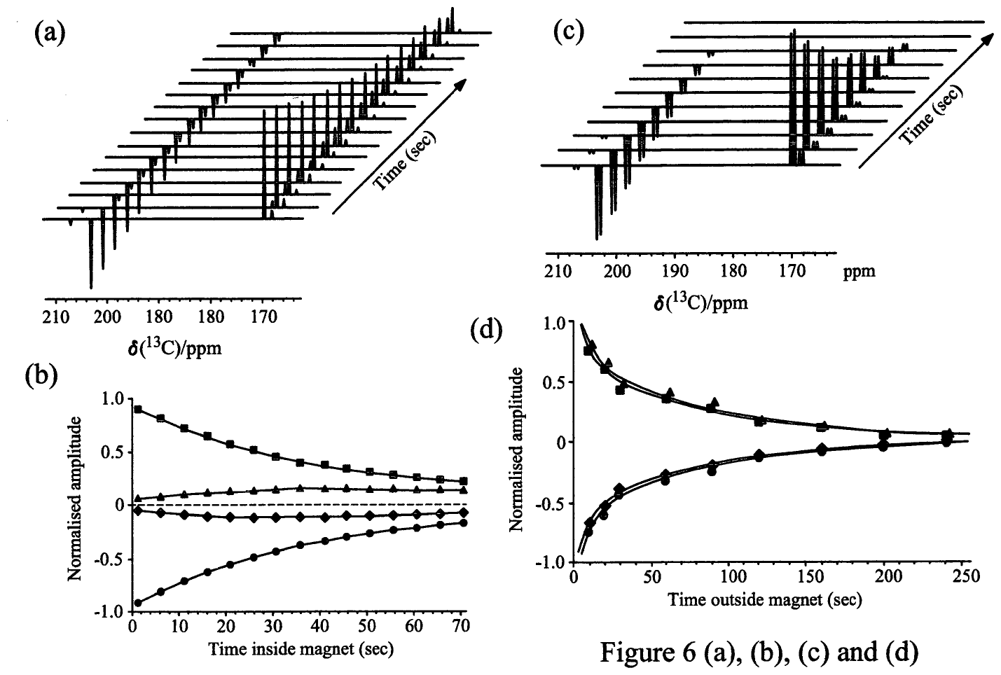

The lifetime of this singlet state was assessed after storage at both low and high field, as detailed in FIG. 6. As expected, essentially exponential signal decays are observed that yield lifetimes of 85.4±8.5 s at low-field and 43.5±0.8 s at 11.7 T. The high-field evolution indicates evidence for substantial cross-relaxation induced polarisation transfer within the spin-system. This relaxation behaviour was probed as a function of catalyst loading to determine whether its presence reduces the lifetime of this state and the effect was found to be very minimal. In the limit a value of 85.4 s is indicated which is 311% better than the individual T₁'s of the ¹³C responses in 1 and 2.

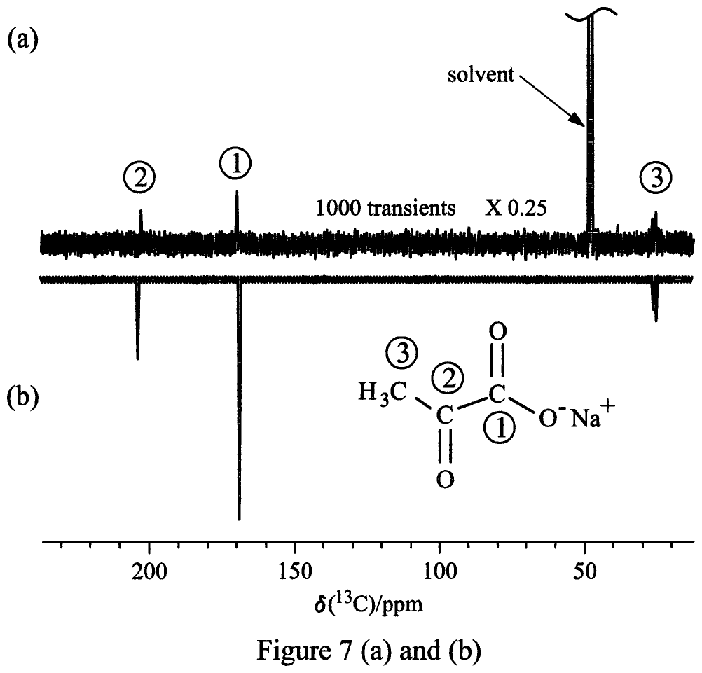

Up to this point, we have been detecting specifically ¹³C labelled isotopologues of pyruvate. FIG. 7 shows the single scan response of an unlabeled sample of 4 (1.5 mg in 0.6 ml of CD₃OD). The resulting S/N ratio was 280 for the C₁ resonance with signals for C₂ and C₃ being clearly visible. We note now that the corresponding C₃ signal was also visible in the spectra of samples 1-3 referred to earlier. This further highlights the context of the successful hyperpolarisation of pyruvate. For comparison, the corresponding ¹³C relaxation times and polarisation levels are detailed in Table 1. Within error, the lifetimes of the C₁ and C₂ Zeeman polarisations are the same across the samples.

In summary, we have illustrated a novel approach to hyperpolarise pyruvate directly by the SABRE hyperpolarisation technique for the forms of pyruvate 2-4

In order to further optimise this process the sulfoxide ligand can be changed with suitable representative examples being diphenlysulfoxide, dibutylsulfoxide, dibenzysulfoxide, phenylmethylsulfoxide, phenylethylsulfoxide, phenylvinyl sulfoxide, dimesityl sulfoxide.

In addition, the NHC can be varied according to the earlier figure in conjunction with the sulfoxide to further optimise this process for a given substrate.

In order to illustrate the effect of DMSO in promoting the SABRE enhancement of a nitrogen containing heterocycle we select d₂-4,6-nicotinamide as the target. When it is hyperpolarised as a 6 mM solution of [Ir]d₁₆-IMesCl with a 21 fold d₂-4,6-nicotinamide excess in 30% d₆-ethanol and 70% D₂O at 313 K a 278 fold signal gain can be seen. When this is repeated with 2 μl of dimethylsulfoxide, the signal gain increases to 370 fold. This gain is dependent on the catalyst with h₁₆-IMesCl returning a 190 fold improvement. In contrast, if imidazole is use as the co-ligand instead of dimethysulfoxide, the signal gain is still 370 fold. Hence in this case, we can conclude that the co-ligands imidazole and dimethylsulfoxide are both able to improve the efficiency of SABRE over the substrate alone.

Further examples include amino acids such as glycine, lactic acid, salicylic acid, glucose, urea, succinate, acetamide and phosphate.

The invention will now be illustrated by way of example only and with reference to the accompanying drawings, in which:

Scheme 1 illustrates the route to hyperpolarisation via SABRE via a metal catalyst;

Scheme 2 illustrates the SABRE hyperpolarization process wherein para-hydrogen (p-H₂) is used to hyperpolarize pyruvate;

FIG. 1 illustrates (a) a single scan hyperpolarized NMR spectrum of 1 (inset) under optimum SABRE-SHEATH condition; and (b) a corresponding single scan thermally polarized spectrum (vertically scaled by 256 times relative to trace a) highlighting the free and bound ¹³C resonances of 1;

FIG. 2 is a plot showing how the ¹³C response of hyperpolarised 1 varies as a function of magnetic field experienced during polarisation transfer, maximum polarisation transfer efficiency (signal intensity) is achieved for an ˜9 mG field;

FIG. 1 illustrates SABRE hyperpolarisation of 1 (a) and 2 (b) at 5 mG mixing field showing enhanced ¹³C signal from individual carbons (a close examination of these NMR spectra reveals the singlet response originating only from the 1.1% of the samples. (c) SABRE NMR spectra of 1 when mixed at Earth's magnetic stray field (˜500 mG), no direct transfer);

FIG. 4 is (a) an energy level diagram for the evolution of the two spin-½ coupled spin system of 3 after hyperpolarization and moving from low (left) to high field (right); and (b) a simulated NMR spectra resulting from 900 and low flip angle excitation;

FIG. 5 illustrates a simulated and experimentally observed spectral pattern of 3 arising from naturally formed singlet states by SABRE mechanism at laboratory field mixing;

FIG. 6 illustrates (a) a high field small flip angle pulse (9 deg); (b) change in measured signal amplitude as a function of low-field storage time showing evolution of the four peak intensities; (c) low field response after storage as measured by 90 degree excitation; and (d) bi-exponential fitting to yield T_{LLS};

FIG. 7 is a ¹³C{¹H} NMR spectra of 4 measured at 11.75 T: (a) thermally polarised spectrum after 1000 signal additions over 16 hours; and (b) single scan hyperpolarised SABRE-SHEATH result after transfer at 9 mG showing the ready detection of all three pyruvate carbon signals as attributed;

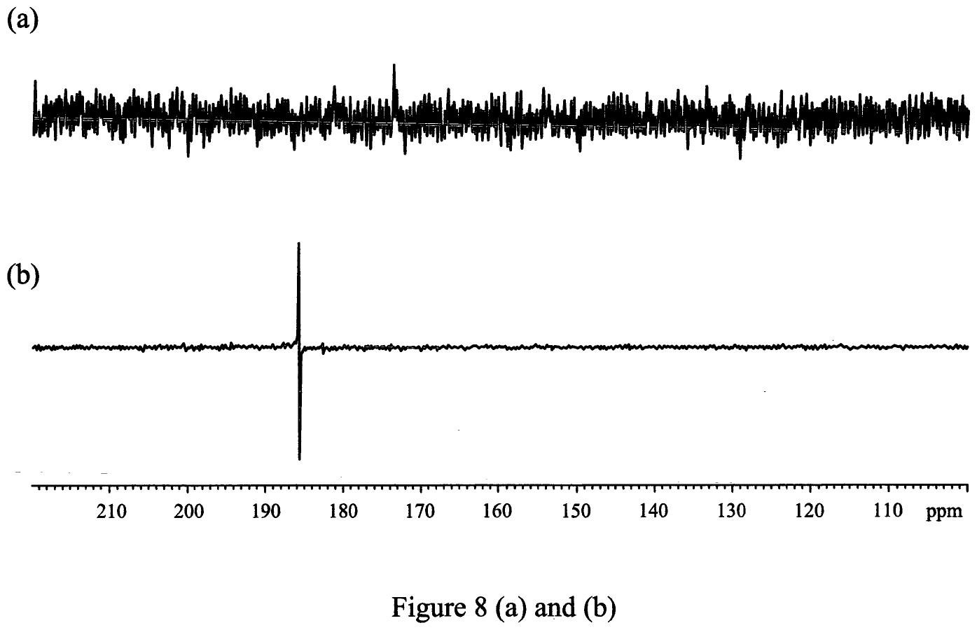

FIG. 8 is a ¹³C NMR of glycine. (a) Boltzmann equilibrium conditions (×32 vertical expansion relative to (b)), (b) SABRE hyperpolarized trace detailing a ˜1000-fold signal enhancement;

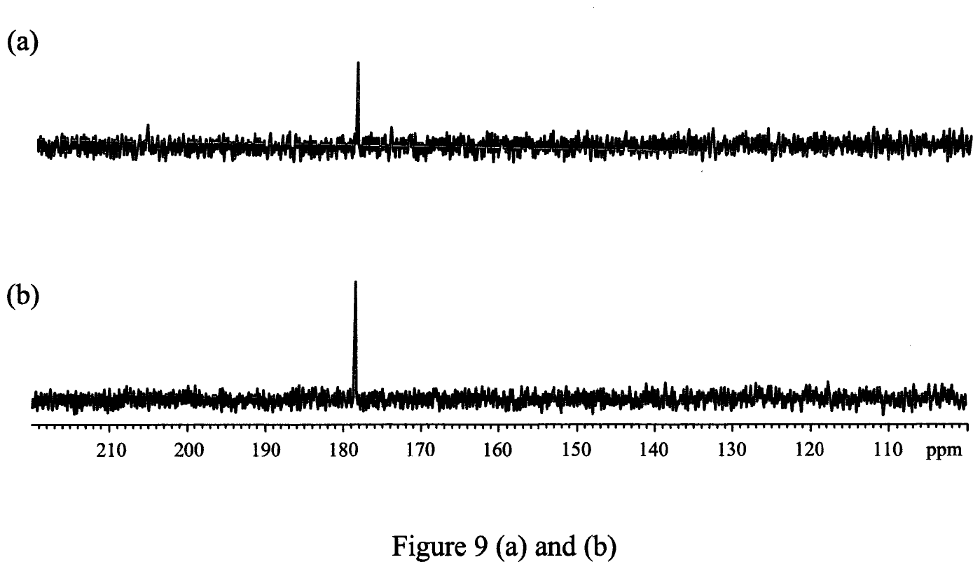

FIG. 9 is a ¹³C NMR of sodium acetate. (a) Boltzmann equilibrium conditions (×2) vertical expansion relative to (b)), (b) SABRE hyperpolarized trace detailing a 4-fold signal enhancement;

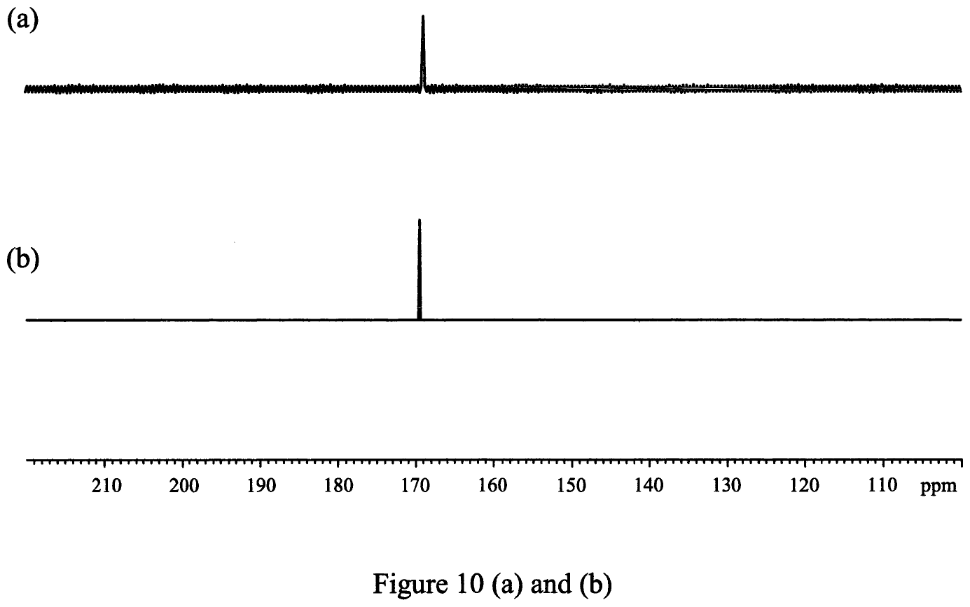

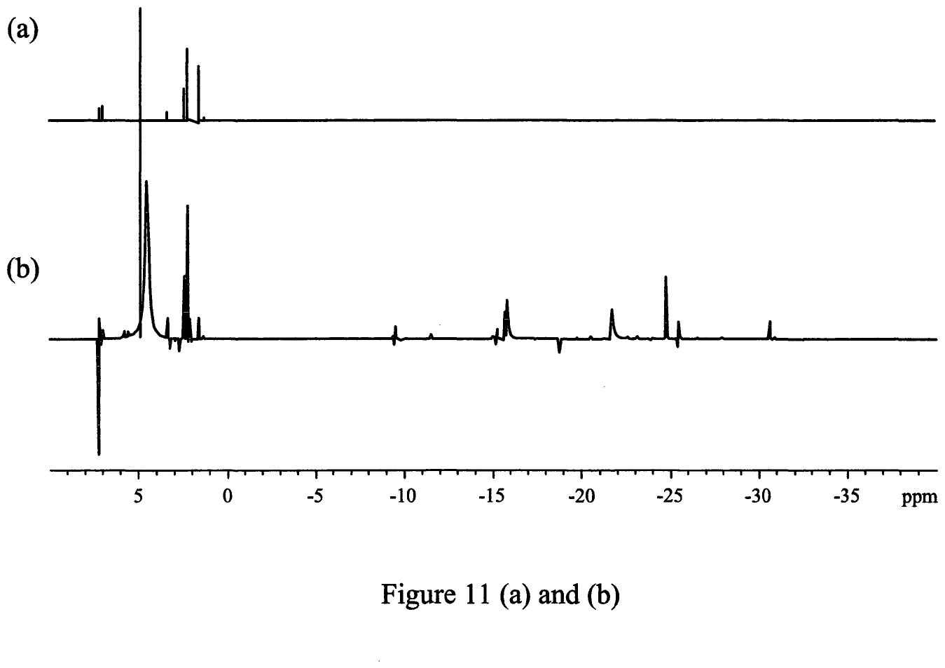

FIG. 10 is a ¹³C NMR of maleic acid. (a) Boltzmann equilibrium conditions (×20 vertical expansion relative to (b)), (b) SABRE hyperpolarized trace detailing a 100-fold signal enhancement; and FIG. 11 is a ¹H NMR of urea. (a) Boltzmann equilibrium conditions (32 scan), (b) 1 scan SABRE hyperpolarized trace signal enhancement.

TABLE 1
Table listing typical hyperpolarization levels and spin orientation
lifetimes under the indicated circumstances for substrates 1-4.

| | | Thermally- | |
|---|---|---|---|
| | | polarised | Hyperpolarised |
| | Net ¹³C | sample | sample lifetimes |
| | polarisation | lifetimes | (T₁ and T_{LLS}) in |
| Substrate | (%) | (T₁) in sec. | sec. |
| 1 | C1: 0.96 | 35.4 ± 0.5 | T₁: 32.5 ± 4.7 |
| 2 | C2: 0.60 | 20.1 ± 0.5 | T₁: 18.2 ± 3.0 |
| 3 | C1: 1.85 | 33.6 ± 0.5 | T_{LLS} (HF): |
| | C2: 1.65 | 21.2 ± 0.4 | 43.5 ± 0.8 |
| | | | T_{LLS} (LF): |
| | | | 85.4 ± 8.5 |
| 4 | C1: 0.55 | 31.4 ± 1.2 | T₁: 28.5 ± 5.5 |
| | C2: 0.35 | 18.6 ± 1.5 | T₁: 15.7 ± 1.9 |
| | C3: 0.22 | 3.3 ± 0.7 | T₁: 3.0 ± 2.5 |

### REFERENCES

1. C. R. Bowers, D. P. Weitekamp, Journal of the American Chemical Society 109, 5541-5542 (1987).
2. J. Natterer, J. Bargon, Progress in Nuclear Magnetic Resonance Spectroscopy 31, 293-315 (1997).
3. R. A. Green, R. W. Adams, S. B. Duckett, R. E. Mewis, D. C. Williamson, G. G. R. Green, Progress in Nuclear Magnetic Resonance Spectroscopy 67, 1-48 (2012).
4. R. W. Adams, J. A. Aguilar, K. D. Atkinson, M. J. Cowley, P. I. P. Elliott, S. B. Duckett, G. G. R. Green, I. G. Khazal, J. Lopez-Serrano, D. C. Williamson, Science 323, 1708-1711 (2009).
5. P. J. Rayner, M. J. Burns, A. M. Olaru, P. Norcott, M. Fekete, G. G. R. Green, L. A. R. Highton, R. E. Mewis, S. B. Duckett, Proceedings of the National Academy of Sciences of the United States of America 114, E3188-E3194 (2017).
6. H. F. Zeng, J. D. Xu, J. Gillen, M. T. McMahon, D. Artemov, J. M. Tyburn, J. A. B. Lohman, R. E. Mewis, K. D. Atkinson, G. G. R. Green, S. B. Duckett, P. C. M. van Zijl, Journal of Magnetic Resonance 237, 73-78 (2013).
7. E. B. Ducker, L. T. Kuhn, K. Munnemann, C. Griesinger, Journal of Magnetic Resonance 214, 159-165 (2012).
8. R. E. Mewis, R. A. Green, M. C. R. Cockett, M. J. Cowley, S. B. Duckett, G. G. R. Green, R. O. John, P. J. Rayner, D. C. Williamson, Journal of Physical Chemistry B 119, 1416-1424 (2015).
9. R. E. Mewis, K. D. Atkinson, M. J. Cowley, S. B. Duckett, G. G. R. Green, R. A. Green, L. A. R. Highton, D. Kilgour, L. S. Lloyd, J. A. B. Lohman, D. C. Williamson, Magnetic Resonance in Chemistry 52, 358-369 (2014).
10. J. F. P. Colell, A. W. J. Logan, Z. J. Zhou, R. V. Shchepin, D. A. Barskiy, G. X. Ortiz, Q. Wang, S. J. Malcolmson, E. Y. Chekmenev, W. S. Warren, T. Theis, Journal of Physical Chemistry C 121, 6626-6634 (2017).
11. D. A. Barskiy, R. V. Shchepin, C. P. N. Tanner, J. F. P. Colell, B. M. Goodson, T. Theis, W. S. Warren, E. Y. Chekmenev, Chemphyschem 18, 1493-1498 (2017).
12. V. V. Zhivonitko, I. V. Skovpin, I. V. Koptyug, Chemical Communications 51, 2506-2509 (2015).
13. M. J. Burns, P. J. Rayner, G. G. R. Green, L. A. R. Highton, R. E. Mewis, S. B. Duckett, Journal of Physical Chemistry B 119, 5020-5027 (2015).
14. R. W. Adams, S. B. Duckett, R. A. Green, D. C. Williamson, G. G. R. Green, Journal of Chemical Physics 131, (2009).
15. A. N. Pravdivtsev, A. V. Yurkovskaya, H. M. Vieth, K. L. Ivanov, R. Kaptein, Chemphyschem 14, 3327-3331 (2013).
16. A. N. Pravdivtsev, A. V. Yurkovskaya, K. L. Ivanov, H. M. Vieth, Journal of Magnetic Resonance 254, 35-47 (2015).
17. N. Eshuis, R. Aspers, B. J. A. van Weerdenburg, M. C. Feiters, F. Rutjes, S. S. Wijmenga, M. Tessari, Journal of Magnetic Resonance 265, 59-66 (2016).
18. K. D. Atkinson, M. J. Cowley, P. I. P. Elliott, S. B. Duckett, G. G. R. Green, J. Lopez-Serrano, A. C. Whitwood, Journal of the American Chemical Society 131, 13362-13368 (2009).
19. K. D. Atkinson, M. J. Cowley, S. B. Duckett, P. I. P. Elliott, G. G. R. Green, J. Lopez-Serrano, I. G. Khazal, A. C. Whitwood, Inorganic Chemistry 48, 663-670 (2009).
20. T. Theis, M. Truong, A. M. Coffey, E. Y. Chekmenev, W. S. Warren, Journal of Magnetic Resonance 248, 23-26 (2014).
21. M. Carravetta, O. G. Johannessen, M. H. Levitt, Physical Review Letters 92, (2004).
22. T. Theis, G. X. Ortiz, A. W. J. Logan, K. E. Claytor, Y. Feng, W. P. Huhn, V. Blum, S. J. Malcolmson, E. Y. Chekmenev, Q. Wang, W. S. Warren, Science Advances 2, (2016).
23. A. M. Olaru, S. S. Roy, L. S. Lloyd, S. Coombes, G. G. R. Green, S. B. Duckett, Chemical Communications 52, 7842-7845 (2016).
24. S. S. Roy, P. Norcott, P. J. Rayner, G. G. R. Green, S. B. Duckett, Angewandte Chemie-International Edition 55, 15642-15645 (2016).
25. S. S. Roy, P. J. Rayner, P. Norcott, G. G. R. Green, S. B. Duckett, Physical Chemistry Chemical Physics 18, 24905-24911 (2016).
26. M. J. Cowley, R. W. Adams, K. D. Atkinson, M. C. R. Cockett, S. B. Duckett, G. G. R. Green, J. A. B. Lohman, R. Kerssebaum, D. Kilgour, R. E. Mewis, Journal of the American Chemical Society 133, 6134-6137 (2011).
27. O. G. Salnikov, K. V. Kovtunov, D. A. Barskiy, A. K. Khudorozhkov, E. A. Inozemtseva, I. P. Prosvirin, V. I. Bukhtiyarov, I. V. Koptyug, Acs Catalysis 4, 2022-2028 (2014).
28. M. Leutzsch, L. M. Wolf, P. Gupta, M. Fuchs, W. Thiel, C. Fares, A. Furstner, Angewandte Chemie-International Edition 54, 12431-12436 (2015).
29. R. V. Shchepin, D. A. Barskiy, A. M. Coffey, B. M. Goodson, E. Y. Chekmenev, Chemistryselect 1, 2552-2555 (2016).
30. C. Godard, S. B. Duckett, S. Polas, R. Tooze, A. C. Whitwood, Dalton Transactions, 2496-2509 (2009).
31. D. J. Fox, S. B. Duckett, C. Flaschenriem, W. W. Brennessel, J. Schneider, A. Gunay, R. Eisenberg, Inorganic Chemistry 45, 7197-7209 (2006).
32. D. Blazina, S. B. Duckett, P. J. Dyson, J. A. B. Lohman, Angewandte Chemie-International Edition 40, 3874-+(2001).
33. S. A. Colebrooke, S. B. Duckett, J. A. B. Lohman, R. Eisenberg, Chemistry-a European Journal 10, 2459-2474 (2004).
34. J. S. M. Samec, J. E. Backvall, P. G. Andersson, P. Brandt, Chem. Soc. Rev. 35, 237-248 (2006).
35. S. E. Clapham, A. Hadzovic, R. H. Morris, Coordination Chemistry Reviews 248, 2201-2237 (2004).
36. M. Patel, R. K. Saunthwal, A. K. Verma, Accounts of Chemical Research 50, 240-254 (2017).
37. J. S. Anderson, J. Rittle, J. C. Peters, Nature 501, 84-+(2013).
38. S. S. Roy, P. Norco, P. J. Rayner, G. G. R. Green, S. B. Duckett, Chem. Eur. J. (in press) doe: 10.1002/chem.201702767 (2017).
39. Lehmkuhl et al “Hyperpolarising Water with Parahydrogen”, ChemPhysChem 10.1002/cphc.201700750

1. A method for the preparation of a hyperpolarised agent, wherein said agent comprises at least one —N⁻, —O⁻ or —S⁻ moiety (each of which may optionally be protonated) and at least one secondary binding site; said method comprising the steps of:
(i) preparing a fluid containing a polarisation transfer precatalyst and parahydrogen;
(ii) separately or simultaneously introducing a co-ligand (L) to interact with the transfer precatalyst to facilitate the formation of polarisation transfer catalyst;
(iii) applying a magnetic field or radio frequency excitation to (ii), such that hyperpolarisation is transferred from parahydrogen to the target molecule when it is bound to the transfer catalyst;
(iv) separately or simultaneously introducing a target molecule, wherein said target molecule contains at least at least one —N⁻, —O⁻ or —S⁻ moiety, in conjunction with a secondary binding to form a hyperpolarised agent;
characterised in that the co-ligand is selected from the group consisting of one or more of a sulfoxide, a thioester, a phosphine, an amine, CO, an isonitrile and a nitrogen heterocycle.

2. A method according to claim 1 wherein co-ligand L is a sulfoxide.

3. A method according to claim 2 wherein sulfoxide co-ligand is an alkylsulfoxide.

4. A method according to claim 3 wherein the alkylsulfoxide is dimethylsulfoxide, diethylsulfoxide, dibutylsulfoxide or methylethylsulfoxide.

5. A method according to claim 2 wherein sulfoxide co-ligand is an arylsulfoxide.

6. A method according to claim 5 wherein the arylsulfoxide is diphenylsulfoxide, dibenzysulfoxide, phenylmethylsulfoxide, phenylvinyl sulfoxide or dimesityl sulfoxide.

7. A method according to claim 1 wherein co-ligand L is varied to optimally hyperpolarise the polarisable molecule by the SABRE effect.

8. (canceled)

9. (canceled)

10. (canceled)

11. A method according to claim 7 wherein the polarisable molecule is characterised by a long lifetime in a low magnetic field.

12. A method according to claim 11 wherein the polarisable molecule has a singlet state lifetime that will be 20 seconds or more.

13. A method according to claim 1 wherein the hyperpolarisable molecule contains spin pairs of appropriate ¹H, ¹³C, ³¹P, ¹⁵N, ²⁹Si or ¹⁹F labels to enable the formation of long-live states (singlet states) between the corresponding spin pairs (e.g. ¹H, ¹³C, ³¹P, ¹⁵N, ²⁹Si or ¹⁹F) within a molecular scaffold that contains appropriate ²H or Cl labelling to extend their lifetime.

14. A method according to claim 1 wherein the magnetic field used in the preparation step is an ultra-low magnetic field.

15. A method according to claim 14 wherein the ultra-low magnetic field is <<1 G (<10⁻⁶ T).

16. A method according to claim 1 wherein the polarisable molecule contains at least one —OH moiety.

17. A method according to claim 16 wherein the polarisable molecule comprises an alcohol moiety, such as methanol, ethanol, butanol, glucose, alkaloids, prostaglandins, or their salts e.g. NaOCH₃; NaOH; or a P—OH group, such as PO(OH)₃ or their salts e.g. PO(OH)₂(ONa), such as those P—OH groups found in DNA or adenosine triphosphate; or acid functionalities, such as HCOOH, CH₃COOH, CH₃CH₂COOH, CH₃COCOOH, or their salts e.g. NaOOCCH₃; and the like.

18. A method according claim 1 wherein the polarisable molecule contains at least one —NH moiety.

19. A method according to claim 1 wherein the polarisable molecule comprises an amine or an amide moiety.

20. A method according to claim 1 wherein the polarisable molecule comprises a primary, secondary or tertiary amine, such as NH₃, NH₂Ph, NH₂CH₂Ph, NH₂CH₂HCH₂CH₂Ph; or an amide, such as NH₂COCH₃ or NH₂CONH₂; and the like.

21. A method according to claim 1 wherein the polarisable molecule contains at least one —SH moiety.

22. A method according to claim 1 wherein the polarisable molecule comprises a thiol or thioamide moiety.

23. A method according to claim 1 wherein the target molecule contains at least one —OH moiety.

24. A method according to claim 1 wherein the target molecule comprises an alcohol moiety, such as methanol, ethanol, butanol, glucose, alkaloids, prostaglandins, or their salts e.g. NaOCH₃; NaOH; or a P—OH group, such as PO(OH)₃ or their salts e.g. PO(OH)₂(ONa), such as those P—OH groups found in DNA or adenosine triphosphate; or acid functionalities, such as HCOOH, CH₃COOH, CH₃CH₂COOH, CH₃COCOOH, or their salts e.g. NaOOCCH₃; and the like.

25. A method according to claim 1 wherein the hyperpolarisation transfer catalyst comprises a metal atom that is iridium with at least one N-heterocyclic carbene (NHC) ligand N-heterocyclic carbene and wherein the (NHC) ligand is selected from: 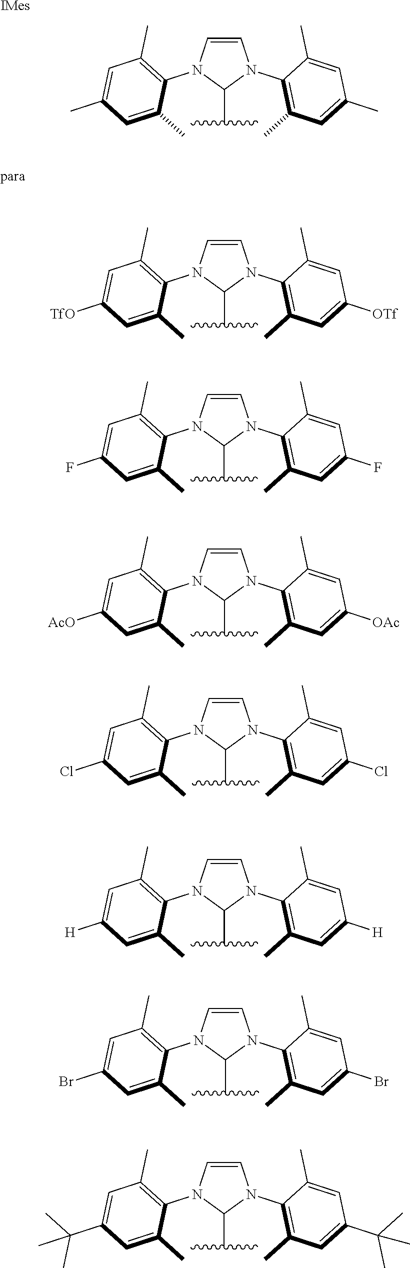 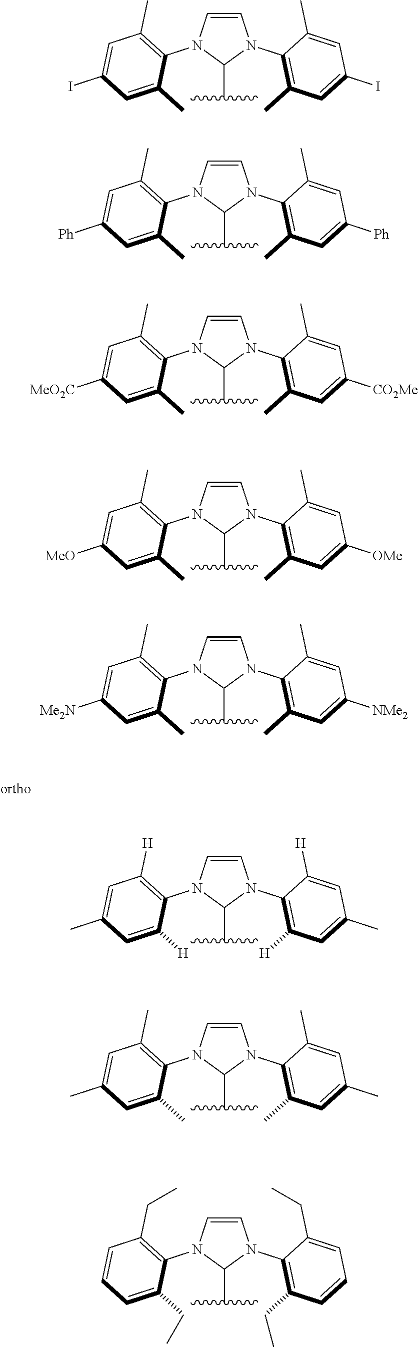 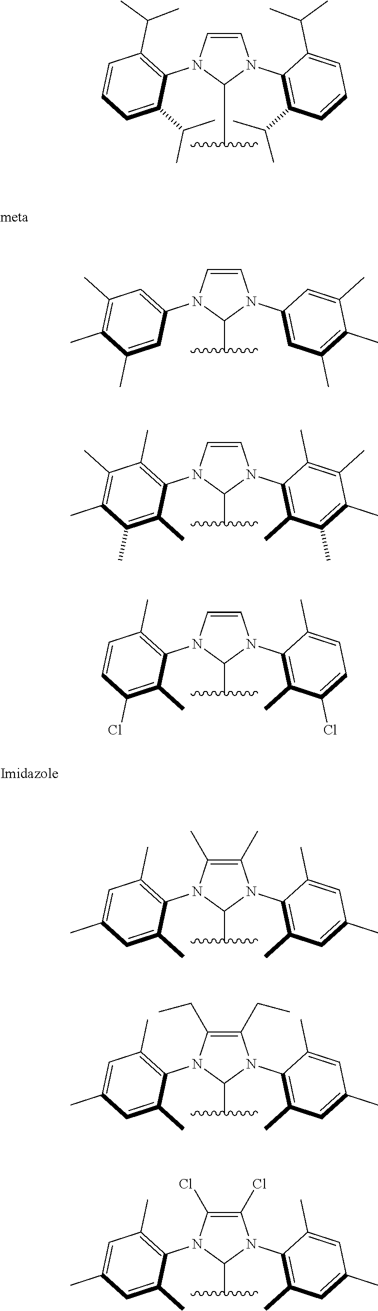 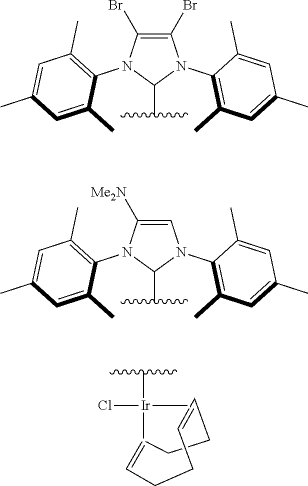 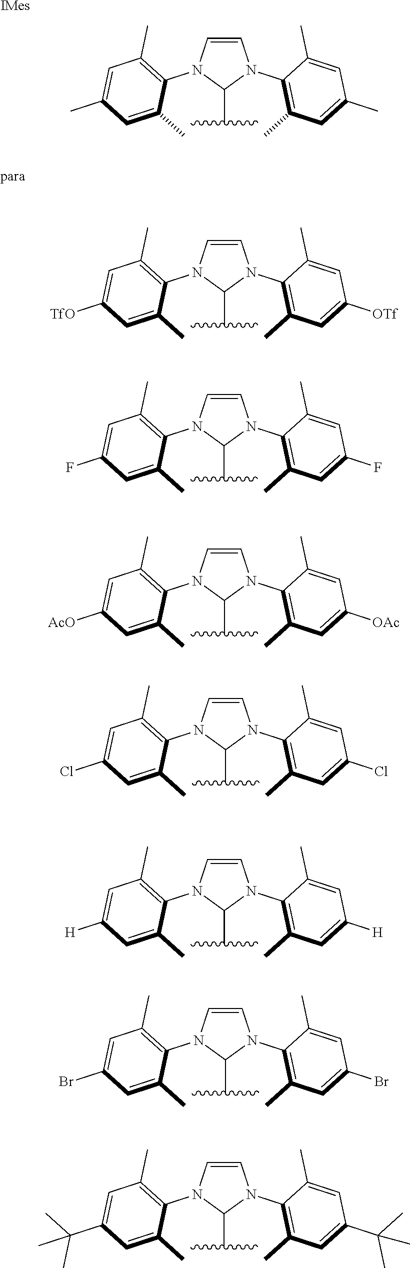 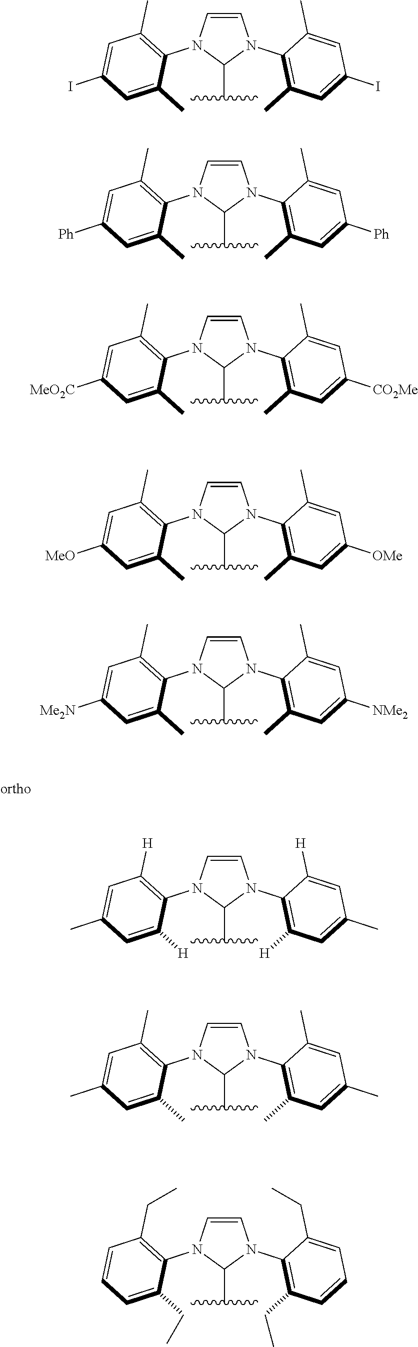 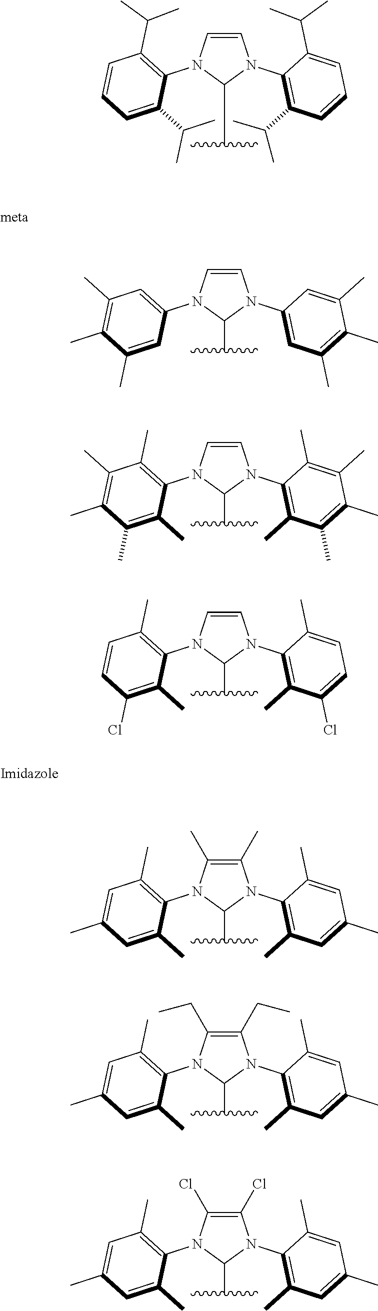 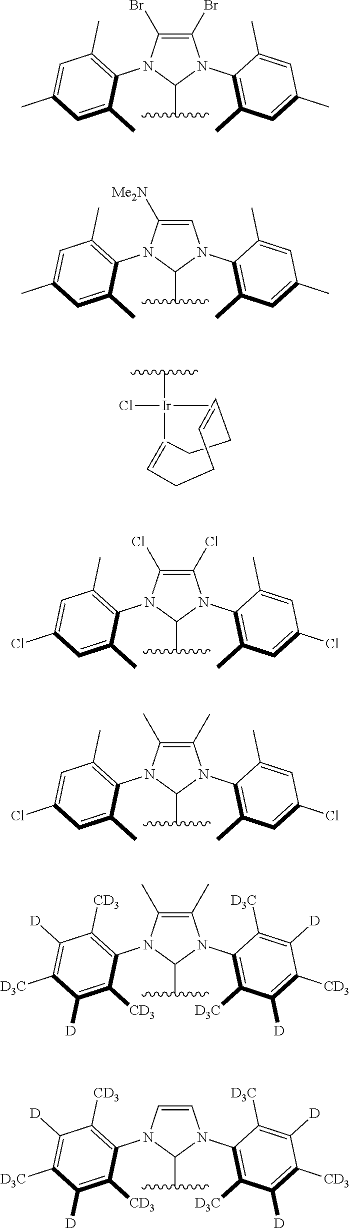 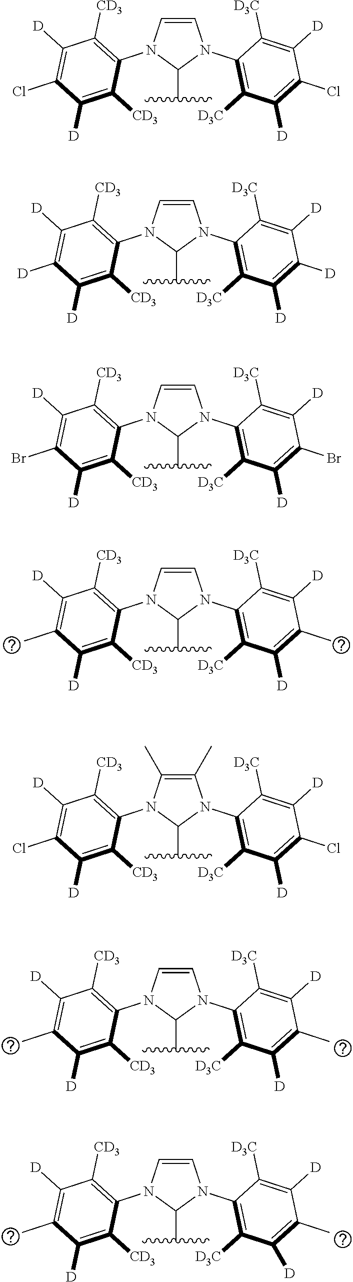 [chemical structure]
